(19) United States
(12) Patent Application Publication
(10) Pub. No.: US 2025/0101528 A1
(43) Pub. Date: Mar. 27, 2025

(54) USES OF CELL-FREE DNA FRAGMENTATION PATTERNS ASSOCIATED WITH EPIGENETIC MODIFICATIONS

(71) Applicant: Centre for Novostics, Shatin (HK)

(72) Inventors: Yuk-Ming Dennis Lo, Homantin (CN); Kwan Chee Chan, Jordan (CN); Peiyong Jiang, Pak Shek Kok (CN); Guanhua Zhu, Ma On Shan (CN)

(21) Appl. No.: 18/883,637

(22) Filed: Sep. 12, 2024

Related U.S. Application Data
(60) Provisional application No. 63/663,564, filed on Jun. 24, 2024.
(60) Provisional application No. 63/539,980, filed on Sep. 22, 2023.

(51) Int. Cl. C12Q 1/6886

(52) U.S. Cl. CPC C12Q 1/6886; C12Q 2600/112; C12Q 2600/154

(57) ABSTRACT

Techniques are provided for using a nucleosome signal pattern of fragmentation at positions around target site(s) for various purposes. For example, the nucleosome signal pattern can be used to determine a methylation level of a target site (e.g., a CpG site). Signals can be associated with nucleosomal patterns of cfDNA molecules within genomic region(s) that are differentially methylated in a target tissue type by having a different methylation level (or multiple levels, e.g., as a pattern) relative to one or more other tissue types (e.g., blood cells). The nucleosome signal pattern can be compared to one or more reference patterns having a known methylation level. Another example approach can determine a level of pathology in a subject. Another example can determine a fractional concentration of DNA of a particular tissue type.

### CROSS-REFERENCES TO RELATED APPLICATIONS

The present application claims priority from and is a non-provisional application of U.S. Provisional Application No. 63/539,980, entitled “Uses Of Cell-Free DNA Fragmentation Patterns Associated With Epigenetic Modifications” filed Sep. 22, 2023 and U.S. Provisional Application No. 63/663,564, entitled “Uses Of Cell-Free DNA Fragmentation Patterns Associated With Epigenetic Modifications” filed Jun. 24, 2024, the entire contents of which are herein incorporated by reference for all purposes.

### BACKGROUND

Cell-free DNA (cfDNA) analysis offers an attractive noninvasive means of detection and monitoring of diseases such as cancers. There are many clues on the nucleosomal origin of plasma DNA. For example, a dominant population of plasma DNA molecules display a predominant size of 166 bp, which coincides with the size of a nucleosome unit (Lo et al. Sci Transl Med. 2010; 2:61ra91). Straver et al. (Straver et al. Prenat Diagn. 2016:36(7):614-621) attempted to use plasma DNA by analyzing the frequency of cfDNA fragments ending within regions 73 bp upstream and downstream of the nucleosome center that was deduced from pooled maternal plasma DNA sequenced reads. Such a value showed a low Pearson correlation of 0.654 with the fetal DNA fraction.

Snyder et al. (Snyder et al. Cell. 2016; 164:57-68) developed a metric, called window protection score, which was defined as the number of molecules spanning a particular genomic window minus those molecules with an endpoint within the window. The window protection score showed wave-like signals across the human genome. Snyder et al. correlated the spacing patterns (distance between peaks) deduced from cfDNA molecules that have a size of 193 to 199 bp with RNA expression datasets. However, the correlation between the median nucleosome spacing in the transcript body and gene expression was only shown to be −0.17. Such a low correlation could not achieve a practically meaningful accuracy for disease diagnosis. In addition, the difference in RNA profiles between the public datasets, which contained 76 cell lines and primary tissues, and the actual bodily organs in a test sample would further reduce the accuracy of cancer detection. Ulz et al. (Ulz et al. Nat Commun. 2019; 10:4666) attempted to use the sequencing coverage around transcription factor binding sites to detect cancers and subtype tumors.

Accordingly, there is a need for more accurate and varied techniques to perform such detections and measurements.

### SUMMARY

Techniques are provided for using a nucleosome signal pattern of fragmentation at positions around target site(s) for various purposes. For example, the nucleosome signal pattern can be used to determine a methylation level of a target site (e.g., a CpG site). Signals can be associated with nucleosomal patterns of cfDNA molecules within genomic region(s) that are differentially methylated in a target tissue type (e.g., of an organ) by having a different methylation level (or multiple levels, e.g., as a pattern) relative to one or more other tissue types (e.g., blood cells). The nucleosome signal pattern can be compared to one or more reference patterns having a known methylation level.

Another example approach can determine a level of pathology in a subject, e.g., detect patients with a pathology (such as cancer); such an example can include determining a tissue type in which the pathology exists. The nucleosome signal pattern at one or more CpG sites can be compared to one or more reference patterns determined from one or more training samples having a known level of the pathology. The CpG sites can be differentially methylated in a target tissue type relative to one or more other tissue types.

Another example can determine a fractional concentration of DNA of a particular tissue type, e.g., one that is differentially methylated at a CpG site. In this example, the nucleosome signal pattern can be compared to one or more reference patterns determined from one or more calibration samples having known fractional concentrations of DNA from the particular tissue type.

One general aspect includes a method for measuring methylation of a target CpG site in a genome of a subject using cell-free DNA molecules. The method can include analyzing a plurality of cell-free DNA molecules from a biological sample of the subject, where analyzing each of the plurality of cell-free DNA molecules includes determining two genomic positions in a reference genome corresponding to both ends of the cell-free DNA molecule. The method can also include determining a nucleosome signal pattern for a genomic region around the target CpG site by: for each genomic position within the genomic region: determining a first amount of the plurality of cell-free DNA molecules that span a window around the genomic position, the window being two bp or greater in length; determining a second amount of the plurality of cell-free DNA molecules that end within the window around the genomic position; and determining a per-position nucleosome signal using the first amount and the second amount, where the genomic region is at least 140 bp in length. The method can also include determining a methylation level of the target CpG site in the genome of the subject based on a comparison of the nucleosome signal pattern to a reference pattern, where the reference pattern is determined from one or more training samples having a known methylation level.

Another general aspect includes a method of analyzing a biological sample to determine a level of a cancer in the biological sample of a subject. The method can include analyzing a plurality of cell-free DNA molecules from the biological sample of the subject, where analyzing each of the plurality of cell-free DNA molecules includes determining two genomic positions in a reference genome corresponding to both ends of the cell-free DNA molecule. The method can also include determining a nucleosome signal pattern for a genomic region around a target CpG site by: for each genomic position within the genomic region: determining a first amount of the plurality of cell-free DNA molecules that span a window around the genomic position, the window being two bp or greater in length; determining a second amount of the plurality of cell-free DNA molecules that end within the window around the genomic position; and determining a per-position nucleosome signal using the first amount and the second amount, where the target CpG site is differentially methylated in a target tissue type relative to one or more other tissue types. The method can also include determining a classification of the level of the cancer for the subject based on a comparison of the nucleosome signal pattern to a reference pattern, where the reference pattern is determined from one or more training samples having a known level of the cancer, and where the level of the cancer is determined for the target tissue type.

Another general aspect includes a method for measuring a fractional concentration of DNA from a first tissue type in a biological sample of a subject. The method can include analyzing a plurality of cell-free DNA molecules from the biological sample of the subject, where analyzing each of the plurality of cell-free DNA molecules includes determining two genomic positions in a reference genome corresponding to both ends of the cell-free DNA molecule. The method can also include determining a nucleosome signal pattern for a genomic region around a target CpG site by: for each genomic position within the genomic region: determining a first amount of the plurality of cell-free DNA molecules that span a window around the genomic position, the window being two bp or greater in length; determining a second amount of the plurality of cell-free DNA molecules that end within the window around the genomic position; and determining a per-position nucleosome signal using the first amount and the second amount, where the target CpG site is differentially methylated in the first tissue type relative to one or more other tissue types in the biological sample. The method can also include determining the fractional concentration of DNA from the first tissue type in the biological sample by comparing the nucleosome signal pattern to a reference pattern, where the reference pattern is determined from one or more calibration samples having known fractional concentrations of DNA from the first tissue type.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 is a diagram illustrating the identification of various molecular characteristics associated with cell-free DNA molecules.

FIG. 2 illustrates example cell-free DNA fragmentation patterns with respect to nucleosome signals surrounding a genomic region of interest.

FIG. 3 shows the example of nucleosomal signal over CTCF-binding sites.

FIG. 4A shows a difference in nucleosome signal patterns for sites having different methylation levels. FIG. 4B shows enhanced wave-like nucleosomal signals surrounding HCC tissue-specific hypermethylated and hypomethylated CpG sites.

FIG. 5A shows percentages of the two types of DMSs located in the compartments A and B of the genome.

FIG. 5B shows a receiver operating characteristic curve (ROC) analysis for the prediction of methylation status of at sites in the training and test sets.

FIG. 6 is a flowchart illustrating a method for determining a methylation level at a site using a nucleosome signal pattern according to embodiments of the present disclosure.

FIGS. 7A and 7B show mean fragmentation scores (normalized nucleosome signals) for HCC-specific hypermethylated and hypomethylated sites for healthy controls and patients with advanced-stage HCC (aHCC).

FIG. 8A shows the mean peak-trough distance for healthy controls and aHCC subjects.

FIG. 8B shows a mean peak-trough distance for various subjects: controls, HBV, early (eHCC), intermediate (iHCC), and advanced-stage HCC (aHCC). FIGS. 8C and 8D show that nucleosomal amplitude was also decreased for HCC-specific hypomethylated CpG sites in HCC patients.

FIGS. 9A-9B and FIG. 10 show an example schematic diagram for normalizing nucleosomal score surrounding CpG sites.

FIGS. 11A-11C show a set of graphs for assessing performance of using cfDNA nucleosomal signal patterns as a biomarker for cancer diagnostics.

FIGS. 12A-12B show cancer detection using nucleosomal signals with alternative normalizations.

FIG. 13A provides the HCC probability of the dataset A-trained SVM model for differentiating various levels of HCC patients from all non-HCC subjects for dataset B. FIG. 13B shows an ROC curve for differentiating HCC patients from all non-HCC subjects.

FIG. 14A provides the HCC probability of the SVM model trained with a dataset using one sequencing protocol for differentiating various levels of HCC patients from all non-HCC subjects for a dataset using a different sequencing protocol. FIG. 14B shows an ROC curve for differentiating HCC patients from all non-HCC subjects.

FIGS. 15A-15B show cancer detection shows (HCC probability) in Dataset B based on a SVM model trained from Dataset A using nucleosomal signals.

FIG. 16 shows a graph that identifies Kaplan-Meier analysis of survival in HBV subjects stratified in two risk groups based on predicted HCC probability shown in FIG. 15A.

FIGS. 17A-17B show a set of graphs for evaluating performance of nucleosomal scores for cancer diagnosis in Dataset C generated using nanopore sequencing.

FIGS. 18A-18C show a set of ROC curves (using cfDNA data in Dataset B as an example) for evaluating the effect of data normalization for nucleosomal score patterns and cancer detection.

FIGS. 19A-19C show ROC curves of HCC detection using nucleosome signal patterns based on differentially methylated sites (DMSs) defined by different criteria of methylation levels between the buffy coat and tumor tissues.

FIGS. 20A-20B show a set of graphs that assess performance of nucleosomal score analysis for random CpG sites.

FIG. 21 shows performance of nucleosome signal patterns (FRAGMA^{XR}) relative to other techniques for Dataset A.

FIGS. 22A and 22B show cancer detection results for lung, breast, and ovarian cancers using nucleosome signal patterns.

FIGS. 23A-23C show probabilistic scores of having cancer predicted depending on tumor DNA fraction in plasma of patients with cancers.

FIG. 24 is a flowchart illustrating a method 2000 analyzing a biological sample to determine a level of a cancer in the biological sample of a subject.

FIGS. 25A and 25B show nucleosomal signal patterns for placenta-specific CpG sites: hypermethylated and hypomethylated.

FIG. 26A shows the mean peak-trough distance for hypermethylated sites for different fetal fractions. FIG. 26B shows the mean peak-trough distance for hypomethylated sites for different fetal fractions. FIGS. 26C-26D show fetal DNA fractions predicted by nucleosomal patterns were well correlated with the actual fetal DNA fraction deduced by a single-nucleotide polymorphism (SNP) based approach.

FIG. 27A shows nucleosomal signal patterns for placenta-specific CpG sites: hypomethylated and hypermethylated. FIGS. 27B-27C show the comparison between actual and predicted fetal DNA fractions in the training and test sets. Actual fractions were deduced from single-nucleotide polymorphism (SNP) analysis.

FIGS. 28A-28B show that donor-derived DNA fractions predicted by nucleosomal patterns were well correlated with the actual donor-derived DNA fraction deduced by single-nucleotide polymorphism analysis, with a Pearson's correlation of 0.99 and 0.97 for training and test sets respectively.

FIGS. 29A-29B show that tumor-derived DNA fractions predicted by nucleosomal patterns were well correlated with the tumor-derived DNA fraction deduced by ichorCNA, with a Pearson's correlation of 0.96 and 0.94 for training and test sets respectively.

FIG. 30 is a flowchart illustrating a method of determining a fractional concentration of DNA from a first tissue type in a biological sample.

FIG. 31 illustrates a measurement system according to an embodiment of the present disclosure.

FIG. 32 shows a block diagram of an example computer system usable with systems and methods according to embodiments of the present disclosure.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to non-tumoral cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue. Various number of regions, sites, or loci can be analyzed, e.g., 50, 100, 200, 500, 1,000, 5,000, 10,000, 50,000, 100,000, 500,000, one million, or more. Various techniques can determine a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%.

A nucleosome (nucleosomal) signal pattern can include values for each genomic position in a region. The value at a genomic position can be a measure of a property of cell-free DNA molecules in a window around the genomic position. Example window sizes are 1 bp, 2 bp, 3 bp, 4 bp, 5 bp, 10 bp, 20 bp, 30 bp, 40 bp, 50 bp, 60 bp, 70 bp, 90 bp, 100 bp, 110 b, 120 bp, 130 bp, 140 bp, 150 bp, 160 bp, 170 bp, 180 bp, 190 bp, 200 bp, etc. or greater or smaller than any of these sizes. A window can be a specified value that is equal to or less than any of these preceding numbers. The value (e.g., a per-position nucleosome signal) at a genomic position can be dependent on a first amount of cell-free DNA molecules that end within the window and/or dependent on a second amount of cell-free DNA molecules that span the window. A sum of the two amounts is the total number of DNA molecules that cover the genomic position. As examples, the value can be a relative amount (e.g., a separation value) of any of these amounts, e.g., a ratio of any of the two of the first amount, the second amount, and the sum of the first amount and the second amount. Such a nucleosome signal without any normalization may be referred to as a raw nucleosome signal or a raw nucleosome signal pattern for a region. A nucleosomal signal can be determined using all cfDNA fragments in a plasma sample, or cfDNA fragments of particular size, e.g. 120-180 bp, where 120 is a lower bound and 180 is an upper bound. Other example lower bounds for the size range are 50 bp, 60 bp, 70 bp, 80 bp, 90 bp, 100 bp, 110 bp, 120 bp, 130 bp, and 140 bp. Other example higher bounds for the size range are 150 bp, 160 bp, 170 bp, 180 bp, 190 bp, 200 bp, 210 bp, 220 bp, 230 bp, and 240 bp.

A “nucleosome score” may refer to a nucleosome signal that is normalized using one or more nucleosome signals from one or more regions (e.g., same region over the site of interest, flank region(s), or one or more regions that are farther away, including on a different chromosome). For example, a statistical value (e.g., an average, mean, median, etc.) can be taken of these nucleosome signals and applied to the nucleosome signal pattern (e.g., divided or subtracted or combinations thereof to each value in the pattern). The result can be a “nucleosome score pattern” including nucleosome scores at a set of positions in a region.

A “normalized nucleosome score” (also referred to as a background-adjusted nucleosome score or just background nucleosome score) may refer to a nucleosome score that is normalized using background (baseline) signals for one or more other regions of a same sample. A given nucleosome score (or value if raw signal is used) at a given position can be normalized using a corresponding nucleosome score (i.e., as same position relative to target site, e.g., CpG site) determined from one or more other regions. Thus, this normalization can be a per position normalization. A normalized nucleosome signal may refer to such normalization of a raw nucleosome signal.

A “distribution-adjusted nucleosome score” may refer to a nucleosome score that is adjusted based on a mean and/or variance (e.g., standard deviation) of a distribution of reference nucleosome signals for the same region or other region(s) of other samples that can be taken as control baseline, such as healthy samples. An example is a z-score or a t-score. Such adjusting (normalization) can be performed on a per position basis. Thus, a mean and variance can be determined for a particular position (using values from other regions) and used to determine the distribution-adjusted value for that given position for a current sample and region(s). Such adjusting can be applied to any of the nucleosome values described herein (e.g., raw signals, nucleosome score, and background-adjusted values). Example distributions defining how the mean and variance are used include the normal distribution, t-distribution, Poisson distribution, Gamma distribution, binomial distribution, Beta distribution, and Cauchy distribution. For a model that generalizes well across cohorts (e.g. a model trained using dataset A to predict on dataset B), normalized nucleosome scores and distribution-adjusted nucleosome scores can provide increased accuracy. In an example, the nucleosomal scores from aggregated regions associated with hypomethylated and hypermethylated CpG sites can be subjected to two steps of data normalization. Randomly selected genomic regions (200,000) are each centered on a CpG site. Sequenced cfDNA molecules from such genomic regions can be aggregated together to determine background nucleosomal scores. Background nucleosomal scores for hypomethylated and hypermethylated CpG sites can be divided by the background nucleosomal scores according to the positions relative to the center CpG, termed normalized nucleosomal scores. Next, on the basis of a set of healthy subjects, the mean values (μ) and standard deviations (δ) of normalized nucleosomal scores for each position relative to the center CpG can be determined. The normalized nucleosomal score (S) could be translated into the nucleosomal z-score by:

$$Nucleosomal z - score = \frac{S - μ}{δ} .$$

For a dataset, half of healthy subjects can be used for determining μ and δ values. Other statistical values besides a mean (e.g., other aggregate statistical values, such as a median or mode) and standard deviation (e.g., other dispersion values) can be determined for other distribution-adjusted nucleosome scores.

Any of the above nucleosome values may collectively be referred to as a nucleosome signal that for a region comprise a nucleosome signal pattern.

“DNA methylation” in mammalian genomes typically refers to the addition of a methyl group to the 5′ carbon of cytosine residues (i.e., 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported.

The “methylation index” for each genomic site (e.g., a CpG site) can refer to the proportion of DNA fragments (e.g., as determined from sequence reads or probes) showing methylation at the site over the total number of reads covering that site. A “methylation status” can refer to whether a particular site is methylated at a particular site of a particular DNA fragment. A “read” can correspond to information (e.g., methylation status at a site) obtained from a DNA fragment. A read can be obtained using reagents (e.g., primers or probes) that preferentially hybridize to DNA fragments of a particular methylation status. Typically, such reagents are applied after treatment with a process that differentially modifies or differentially recognizes DNA molecules depending on their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines. Some embodiments can determine a methylation level without such treatment processes.

The “methylation density” of a region or a set of sites can refer to the number of reads at site(s) within the region (also referred to as a bin) or the set of sites showing methylation divided by the total number of reads covering the site(s) in the region or the set of sites. A region can include one or more sites of interest, including at least 1, 2, 3, 4, 5, 10, 20, 50, 100, 200, 500, and 1,000 sites. The site(s) may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer to the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110: 18910-18915) and by the Pacific Biosciences single molecule real time analysis (Tse et al. Proc Natl Acad Sci USA 2021; 118: e2019768118).

A “methylation levef” is an example of a relative abundance, e.g., between methylated DNA molecules (e.g., at one or more particular sites) and other DNA molecules (e.g., all other DNA molecules or just unmethylated DNA molecules at the one or more particular sites). The amount of other DNA molecules can act as a normalization factor. As another example, an intensity of methylated DNA molecules (e.g., fluorescent or electrical intensity) relative to intensity of all or unmethylated DNA molecules can be determined. The relative abundance can also include an intensity per volume. A methylation level can be determined using a methylation-aware assay such as methylation-aware sequencing or PCR. Example methylation-aware sequencing can include bisulfite sequencing or single molecule techniques, e.g., using nanopores.

A differentially methylated region (DMR) is a genomic region (e.g., set of sites) with different DNA methylation level across two or more biological samples. The different DNA methylation level may be defined by the certain difference in methylation index or density, such as but not limited to 1%, 5%, 10%, 20%, 30%, 40%, 50%, 60%, 70%, 80%, 90%, 95%, 99%, etc. A differentially methylated site (DMS) may be defined in a similar manner.

The term “hypomethylation” can refer to a site or set of sites (e.g., a region) that has below a specified threshold for a methylation level, e.g., at or below 50%, 45%, 40%, 35%, 30%, 25%, or 20% for the methylation level. A site in a genome may be considered unmethylated if the methylation level is below a threshold. The term “hypermethylation” can refer to a site or set of sites (e.g., a region) that has above a specified value for a methylation level, e.g., at or above 95%, 90%, 80%, 75%, 70%, 65%, or 60% for the methylation level. A site in a genome may be considered methylated if the methylation level is greater than a threshold.

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” and a measured or known fractional concentration of the clinically-relevant DNA (e.g., DNA of particular tissue type). The calibration value can be determined from a reference pattern, e.g., where the calibration value is a peak-to-trough distance, as determined for a calibration sample, for which the fractional concentration of the clinically-relevant DNA is known. The calibration data points may be defined in a variety of ways, e.g., as discrete points or as a calibration function (also called a calibration curve or calibration surface). The calibration function could be derived from additional mathematical transformation of the calibration data points. The calibration value can be part of a calibration reference pattern, e.g., a nucleosome reference pattern that is determined from one or more calibration samples known to have a similar fractional concentration. The fractional concentration can be determined in various ways, e.g., using a tissue-specific allele, a tissue-specific methylation value or pattern, and a size distribution of a sample with a known fractional concentration.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis.

A “separation value” corresponds to a difference or a ratio involving two values, e.g., two fractional contributions or two methylation levels. A separation value is an example of a parameter. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio. A separation value can be compared to a threshold to determine whether the separation between the two values is statistically significant. A per-position nucleosome signal can be a separation value.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “level of pathology” can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). Pregnancy can be considered a pathology. A heathy state of a subject can be considered a classification of no pathology.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, or one million parameters. An ML model can be generated using sample data (e.g., training samples) to make predictions on test data. Various number of training samples can be used, e.g., at least 10, 100, 1,000, 5,000, 10,000, 50,000, 100,000, or at least 200,000 training samples. One example is an unsupervised learning model. Another example type of model is supervised learning that can be used with embodiments of the present disclosure. Example supervised learning models may include different approaches and algorithms including analytical learning, statistical models, artificial neural network (e.g. including convolutional and/or transformer layers), boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ordinal classification, data pre-processing, handling imbalanced datasets, statistical relational learning, or Proaftn (a multicriteria classification algorithm), or an ensemble of any of these types. The model may include linear regression, logistic regression, deep recurrent neural network (e.g., long short-term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

In this disclosure, we develop new approaches of using a nucleosome signal pattern of fragmentation at positions around target site(s) for various purposes. For example, the nucleosome signal pattern can be used to determine a methylation level of a target site (e.g., a CpG site). Other examples can make use of signals associated with nucleosomal patterns of cfDNA molecules within genomic region(s) that are differentially methylated in a target tissue type (e.g., of an organ) by having a different methylation level (or multiple levels, e.g., as a pattern) relative to one or more other tissue types (e.g., blood cells). Such nucleosomal patterns can be referred to as CpG-associated cfDNA nucleosomal patterns. The tissue types can include but not limited to the blood cells, liver, lungs, bladder, kidney, spleen, pancreas, heart, stomach, intestines, etc. The nucleosome signal pattern can be compared to one or more reference patterns having a known methylation level.

Another example approach can determine a level of pathology in a subject, e.g., detect patients with a pathology (such as cancer); such an example can include determining a tissue type in which the pathology exists. In this example, the nucleosome signal pattern can be compared to one or more reference patterns determined from one or more training samples having a known level of the pathology. Examples results assess the diagnostic performance of distinguishing between patients with and without hepatocellular carcinoma (HCC), based on nucleosomal signals derived from differentially methylated regions between HCC tumoral tissues and buffy coat cells.

Another example can determine a fractional concentration of DNA of a particular tissue type, e.g., one that is differentially methylated at a site. In this example, the nucleosome signal pattern can be compared to one or more reference patterns determined from one or more calibration samples having known fractional concentrations of DNA from the particular tissue type. Such techniques do not require a tissue-specific allele, and thus do not require an additional, initial measurement to determine a tissue-specific allele or other tissue-specific marker. For example, a biopsy of a tumor is not required to identify a tumor-specific marker such as a tumor-specific allele.

Such differences in methylation of a tissue type can cause effects of fragmentation across a long distance, e.g., over one or more nucleosomes. A fragmentomics-based methylation analysis can include an extended region harboring multiple nucleosomes. Using a nucleosome signal pattern including fragmentation over multiple nucleosomes can provide increased accuracy and/or alternative ways to perform such measurements that may be combined with other techniques. Such measurements can include methylation levels, level of a pathology, and a fractional concentration of DNA from a tissue type (e.g., a tumor tissue type). For example, as DNA methylation patterns can be preferentially altered in diseased cells (e.g., tumor) compared with other types of cells (e.g. hematopoietic cells), the measure of methylation-dependent nucleosomal signals can enable cancer detection. Such cancer detection can achieve improved accuracy relative to other techniques, thereby allowing fewer false positives and false negative and enabling early therapeutic intervention.

The signals associated with nucleosomal patterns can be defined in various ways, e.g., as the ratio of the number of cfDNA molecules spanning a genomic window (e.g., one of various sizes described herein, such as 140 bp) relative to the number of cfDNA molecules ending within such a window. Nucleosomal signal can be determined using all cfDNA fragments in a plasma sample, or cfDNA fragments of particular size, e.g. 60-120 bp, 80-140 bp, 100-160 bp, 120-180 bp, 140-200 bp, et al. Such signals can also be referred to as nucleosomal signals, which can be methylation-dependent. An advantage of the use of nucleosomal signals for informing methylation changes can eliminate the requirement for processes that differentially modify or differentially recognize DNA molecules depending on their methylation status (e.g., bisulfite treatment) that would degrade DNA drastically (Grunau et al. Nucleic Acids Res. 2001; 29, E65). Moreover, the use of nucleosomal signals can harness other types of epigenetic modifications that would affect the nucleosomal patterns, potentially improving the detection power. And such techniques can be combined with other techniques, e.g., to form an ensemble model, for determining methylation of a site, a level of pathology, or a fractional concentration of cell-free DNA from a particular tissue type.

Various normalizations can be performed resulting in a class of nucleosome signals that can be used in the various applications described herein.

As example results below, we employ a deep-learning model to classify whether a CpG site is HCC-specific hypomethylated or hypermethylated, achieving an area under a receiver operating characteristic curve (AUC) of 0.87. Compared with subjects without cancer, patients with hepatocellular carcinoma (HCC) showed reduced amplitude of nucleosomal patterns, with a gradual decrease over tumor stages. As another example result, the use of nucleosomal patterns associated with differentially methylated CpG sites detected patients with hepatocellular carcinoma with an AUC of 0.93 using a machine learning model. We further validated the cancer detection approach in an independent population cohort. As another example result, a machine learning model (e.g., a regression function) using nucleosomal patterns quantitatively measured fractional concentration of DNA from a first tissue type (e.g., fetal DNA fraction in pregnant women), showing a good concordance (Pearson's r=0.85) with single-nucleotide polymorphism analysis.

This disclosure reveals the interplay between nucleosomal signals and methylation status. Example implementation can exploit methylation-associated nucleosomal patterns to inform tissue-of-origin of cfDNA molecules, such as cancer detection. This approach can incorporate methylation signals without bisulfite sequencing, opening new possibilities of the use of cfDNA sequencing for diagnosis.

In the description below, we first explore whether those CpG sites also show differential fragmentation signal across a genomic region. We then assess the diagnostic performance of distinguishing between patients with and without hepatocellular carcinoma (HCC), based on nucleosomal signals derived from differentially methylated regions between HCC tumoral tissues and buffy coat cells. In addition, the nucleosomal signals related to the tissue of origin were validated in pregnant women.

### I. Relationship Between Nucleosome Signal and Methylation

This disclosure describes processes and techniques that relate a nucleosome signal pattern to a methylation, e.g., a methylation level at a site in the genome. The methylation status of a given site for a given cell can statistically affect the way the DNA is cut by enzymes over a long range (e.g., for length described for genomic regions herein) relative to the site, e.g., spanning one or more nucleosomes. Such changes in fragmentation affect the nucleosome signal at each of the positions in a genomic region, thereby allowing a nucleosome signal pattern to be used to detect the underlying causes of the fragmentation change. Such changes in methylation can occur in various tissue types (e.g., fetal/pregnancy and tumor). Aspects of fragmentation analysis, including nucleosome signal pattern, are now discussed.

### A. Cell-Free DNA Fragmentomics and Methylation

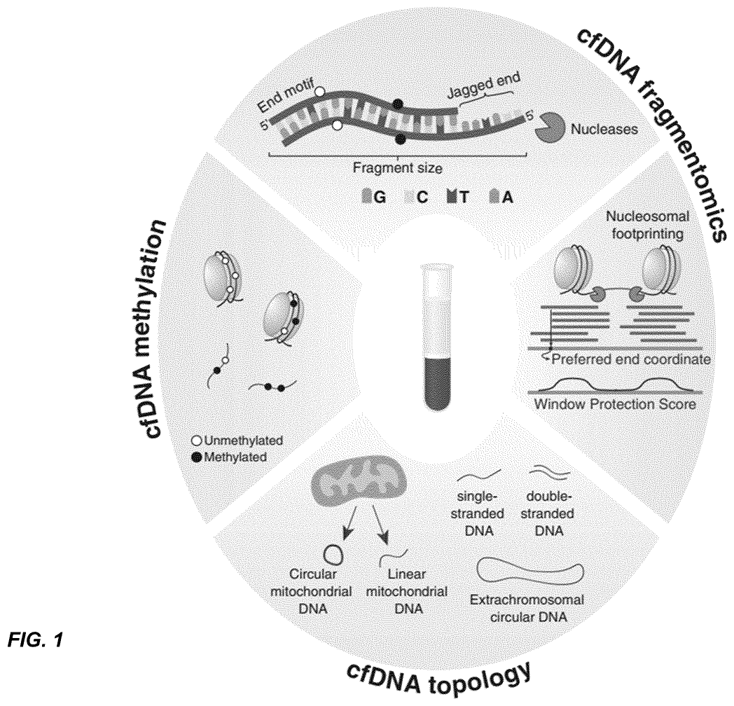

FIG. 1 is a diagram illustrating the identification of various molecular characteristics associated with cell-free DNA molecules. FIG. 1 shows different biomarkers in cell-free DNA including the methylation (e.g., methylation patterns, such as locations of hypermethylation and hypomethylation, global hypermethylation, etc.), fragmentomics (e.g., end motifs, jagged ends, fragments sizes, preferred end coordinates, window protections scores, and nucleosome signal pattern), and topology (e.g., circular mitochondrial DNA, linear mitochondrial DNA, extrachromosomal circular DNA) that can be used separately or in combination.

### B. Nucleosome Signal Pattern

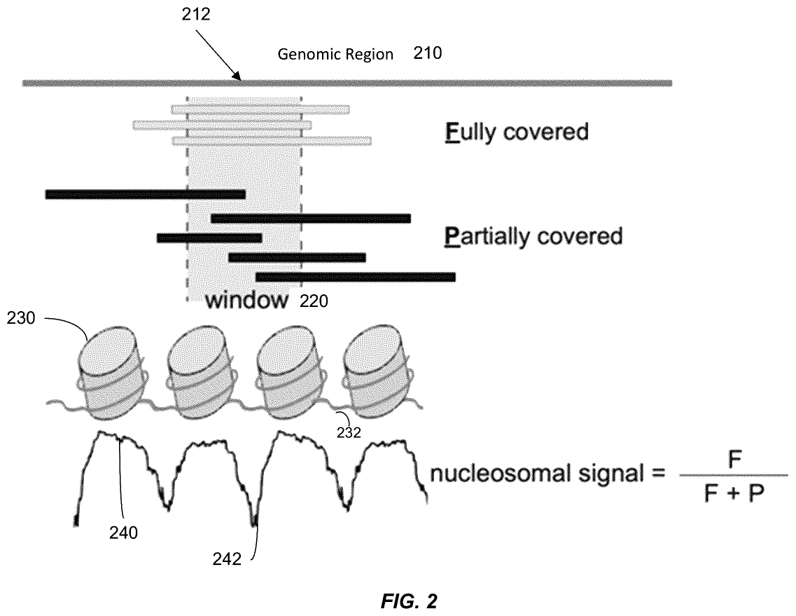

FIG. 2 illustrates an example of cell-free DNA fragmentation patterns with respect to nucleosome signals surrounding a genomic region 210 of interest. The nucleosomal signal for a genomic position can be measured using a genomic window centered on the genomic position (e.g., position 212) with a specified width (e.g., 140 bp). In various embodiments, the window sizes of 10 bp, 20 bp, 30 bp, 40 bp, 50 bp, 60 bp, 70 bp, 80 bp, 90 bp, 100 bp, 110 b, 120 bp, 130 bp, 140 bp, 150 bp, 160 bp, 170 bp, 180 bp, 190 bp, 200 bp, etc. can be used, or any values smaller than or greater than these example sizes.

The numbers of sequenced cfDNA fragments fully (222) and partially (224) covering the window can be determined, respectively. The fraction of the sequenced fragments that fully cover the window is referred to as a nucleosomal signal. As shown in FIG. 2, the number of cfDNA fragments that fully cover the window (denoted by ‘F’) and those that partially cover the window (denoted by ‘P’) were calculated, respectively. The value of F/(F+P) can be used to reflect the nucleosomal signal.

The DNA nucleases would preferentially cut at a linker DNA 232 between two nucleosomes 230 to generate cfDNA fragments. When the center of a window being analyzed corresponds to the dyad of a nucleosome, the value of F/(F+P) would be a peak value 240. When the center of a window being analyzed corresponds to the linker DNA 232 between two nucleosomes, the value of F/(F+P) could be a trough value 242. The nucleosomal signal can be determined for each genomic position of genomic region 210, e.g., for positions covered by sequenced cfDNA molecules. This value (F/(F+P)) can be used as the nucleosomal signal. Other types of separation values can be used, e.g., P/(F+P), P/(F−P), F/(F−P), P−F, F−P, F/P, or P/F. A per-position nucleosome signal can be determined using any of these separation values that use such amounts.

If the position on the genome is highly protected (e.g., in dyad), there are more fully-covered fragments, with the nucleosomal signal being high. If the position is naked (less protected) and highly degraded, then the signal is low. FIG. 2 shows this pattern exists. The peak of the signal is in the same position on the nucleosome.

The fragmentation in serum and urine should be similar to that in plasma as the DNA fragments are the same but collected in a different manner. Other samples besides plasma and serum should show similar fragmentation, as such fragmentation would still result mostly from the same enzymatic process for cutting DNA, and thus the same effects of methylation at a given site would exist.

### C. Normalization Using Nucleosome Signal Values

Some embodiments can correct for inter-sample variation by performing a normalization. The normalization of signals in a region can use signals from the region itself or outside the region, e.g., flank regions that are adjacent to the region. The signals from outside the region can be from anywhere else in the genome and can be any number of external region(s) (e.g., 2, 3, 4, 5, and more) with each being of various lengths, such as 1 kb, 5 kb, 10 kb, 50 kb, 1 Mb, and more. Such a normalization signal can be referred to as a background signal. Certain sites of a region can be used or all the sites in a region can be used. A statistical value (e.g., a mean) across all positions or on a per-position basis can be used to normalize any given nucleosome signal, e.g., by dividing or subtracting.

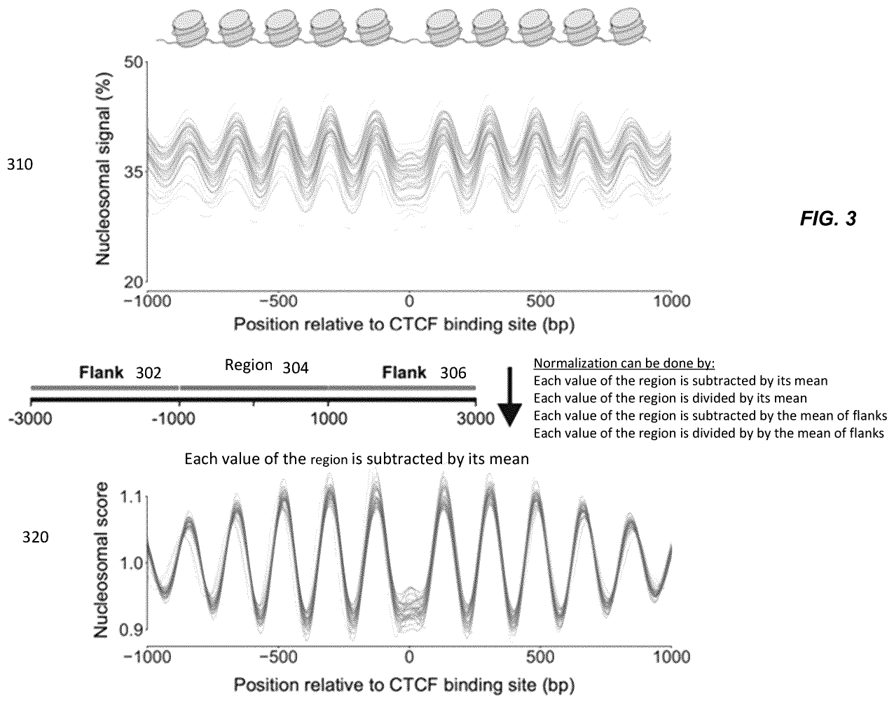

FIG. 3 shows the example of nucleosomal signal over CTCF-binding sites, which have strongly positioned nucleosomes. FIG. 3 shows normalization of nucleosome signals within a region using the mean nucleosomal signal of this region. Plot 310 shows nucleosome signals before normalization, and plot 320 shows nucleosome signals after a region-level normalization that is applied to each signal value, as opposed to a per-position normalization, which is described later. Thus, a region-level statistical value may be used.

The profile of nucleosomal signals was obtained from plasma DNA of healthy subjects. The horizontal axis is the position relative to CTCF binding sites. The vertical axis is the nucleosomal signal or the nucleosomal score (normalized signal). Each line corresponds to a different sample. For a position at a given line, the value is determined as a summation across a set of CTCF bindings sites. Each sample consistently showed the patterns similar to that shown in FIG. 3, as is evidenced by plots 310 and 320.

To minimize the inter-sample variability in terms of nucleosomal signals, one can perform the normalization of nucleosomal signals using the values (e.g., statistical values such as mean value or median value) of nucleosomal signals (e.g., preposition nucleosome signals) derived from the region itself and/or other region(s) (e.g., flank region(s)). The normalized nucleosomal signal can be termed as nucleosomal score. In one example, the nucleosomal signals across a genomic region of interest subtracted by the mean value of nucleosomal signals in this genomic region and/or one or more other regions can be considered as nucleosomal scores (normalized signal). In another example, dividing can be used in the signal normalization step, e.g., dividing by the statistical value or other baseline.

As can be seen, the variation in plot 320 is much smaller than the variation in plot 310. Such a reduction in variation can provide greater accuracy for comparing reference nucleosome signal patterns, which can be used for various purposes as described herein.

In another example, the flank regions are upstream and downstream 2-kb windows, which are used as the normalizing factor. The mean or other statistical value of the flank region(s) can be used to normalize the curve. Such normalizations can provide normalized signals that are more comparable across different samples. Using this normalized score (normalized signal value) at each position, we can calculate the nucleosome signal pattern along the genome.

### II. Determining Methylation Level of a Site in a Genome

The nucleosome signal pattern can detect deviations in the fragmentation pattern caused by different methylation levels. Different reference patterns can be determined for different methylation levels. A comparison of a measured nucleosome signal pattern to one or more reference patterns having a known methylation level (e.g., as measured from samples where methylation is determined using another technique, such as bisulfite treatment) can provide a measurement of the methylation level for a site of a genome in a new sample. Such a measurement using a nucleosome signal pattern can avoid using a bisulfite treatment, which can degrade the DNA resulting in fewer usable DNA fragments.

### A. Methylation Level Determined in General

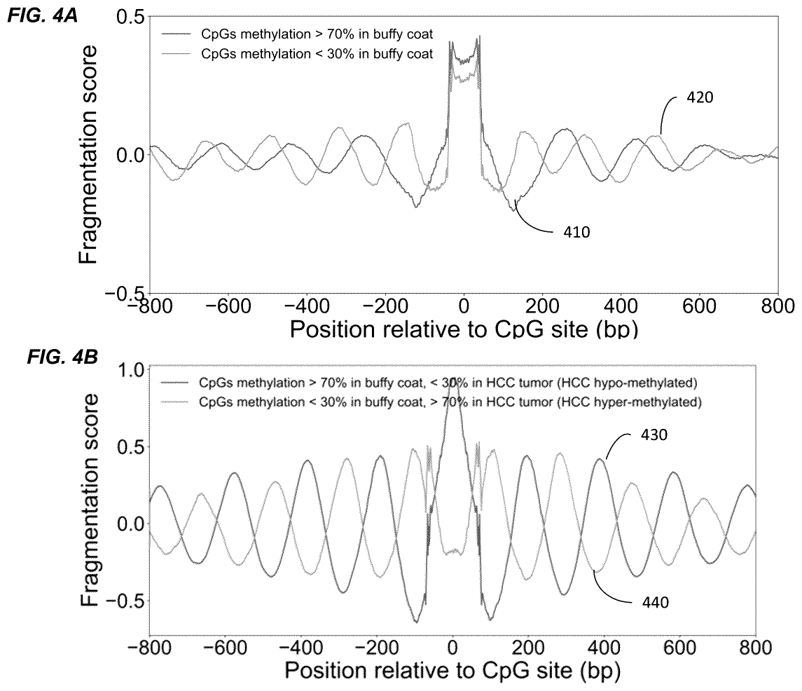

FIG. 4A shows a difference in nucleosome signal patterns for sites having different methylation levels. The horizontal axis shows the position relative to the CpG site. As shown, the region around the CpG site is +/−800 bp (˜1,600 total width). Other sized regions are described herein, such as an overall width (length) of greater than or less than 400, 500, 600, 700, 800, 900, 1,000, 1,100, 1,200, 1,300, 1,400, 1,500, and more etc. The vertical axis shows the nucleosomal score at each position (normalized nucleosome signal pattern) in a region around CpG sites. The region may include one or more CpG sites separate from the target CpG site at position 0.

Nucleosome signal pattern 410 corresponds to CpG sites that have a methylation density greater than 70% (range: 70% to 100%, median: ˜85%) in buffy coat cells. The line of nucleosome signal pattern 410 corresponds to a set of hypermethylated regions using pooled cfDNA data. Nucleosome signal pattern 420 corresponds to CpG sites that have a methylation density less than 30% (range: 0% to 30%, median: ˜15%) in buffy coat cells. The line of nucleosome signal pattern 420 corresponds to a set of hypomethylated regions.

As shown, the two nucleosomal signal patterns (410 and 420) differ. In one example, a measured nucleosome signal pattern that is more similar to nucleosomal signal pattern 410 can be classified as hypermethylated. In another example, a measured nucleosome signal pattern that is more similar to nucleosomal signal pattern 420 can be classified as hypomethylated. Other classifications for different methylation levels can also be used, e.g., intermediate or numerical values, such as ranges.

A predominance (e.g., 80% or higher) of the cell-free DNA in plasma is from the hematopoietic cells. Thus, the nucleosome methylation signal pattern from plasma could be used to detect the overall methylation level at a site in a sample.

Hypomethylation could be defined as, but not limited to, below 10%, below 20%, below 30%, below 40%, below 50%, etc. Hypermethylation could be defined as, but not limited to, over 40%, over 50%, over 60%, over 70%, over 80%, over 90%, etc. The threshold could be a range, for example, but not limited to, 20%-30%, 30%-40%, 40%-50%, 60%-70%, 70%-80%, etc. In FIG. 4A, the hypomethylated and hypermethylated CpG sites were defined as CpG sites with methylation levels of <30% and >70%, respectively, based on bisulfite sequencing results of buffy coat samples (Lun et al. Clin Chem. 2013; 59(11):1583-1594; Tse et al. Proc Natl Acad Sci USA. 2021; 118(5):e2019768118).

As shown in FIG. 4A, cfDNA molecules relative to hypomethylated CpG sites (denoted as position 0) were aligned to determine nucleosomal scores for each genomic position surrounding the CpG sites. Similarly, cfDNA molecules relative to hypermethylated CpG sites (denoted as position 0) were aligned to determine nucleosomal scores for each genomic position surrounding the CpG sites. The nucleosomal scores generally showed wave-like signals surrounding both the hypomethylated and hypermethylated CpG sites. However, nucleosomal score patterns were distinct between hypermethylated and hypomethylated CpG sites in healthy subjects.

There are some offsets (phase differences) in terms of locations related to the peaks and troughs between hypermethylation- and hypomethylation-associated nucleosomal scores. Accordingly, a difference between the two signal patterns is the phase, e.g., the position where the peaks (maxima) and troughs (minima) are. This phase difference can be seen as a shift in the position of the maxima/minima for two signals. The comparison could provide a relative phase difference of the measured signal pattern to one of the reference patterns, where the phase difference can correlate to a methylation level. The reference pattern could also be a sine or cosine of a specified phase relative to the target site (i.e., 0 in FIG. 4A), where the phase difference could be determined for different signal patterns having a known methylation level at the site (or set of sites having a similar methylation level, e.g., all being hypomethylated or all hypermethylated). Accordingly, the phase difference can be measured in a variety of ways.

### B. Methylation Level Determined for Tissue-Specific Sites

A methylation level can also be determined for a particular tissue type at a particular site, even when cell-free DNA from that tissue type is usually a minority in the sample, which is a cell-free mixture of DNA from a plurality of tissue types. This can be done by using sites that are generally hypomethylated in other tissues (e.g. hematopoietic cells) but can be hypermethylated in the particular tissue type (e.g. liver tumor). In this way, any variation in the pattern can be attributed to the methylation level at the site or set of sites.

FIG. 4B shows enhanced wave-like nucleosomal signals surrounding HCC tissue-specific hypermethylated (440) and hypomethylated CpG sites (430). HCC was used as the specific tissue type, but healthy tissue types can also be used. In this example, HCC tumor-specific hypomethylated sites were defined by those CpG sites with a methylation density of below 30% in the HCC tissues but over 70% in the buffy coat samples.

Surprisingly, as shown in FIG. 4B, when one focused on CpG sites showing tissue-specific DNA methylation, the oscillations between peaks and troughs of nucleosomal scores were much enhanced. In this analysis, we analyzed 118,544 HCC-specific hypermethylated CpG sites (e.g. methylation level>70% in HCC tumor, and <30% in buffy coat) and 842,892 HCC-specific hypomethylated sites (e.g. methylation level<30% in HCC tumor, and >70% in buffy coat). The oscillating patterns appeared to be out of phase between HCC-specific hypomethylation and hypermethylation. These results suggested that the methylation patterns could affect the nucleosomal signals of cfDNA molecules.

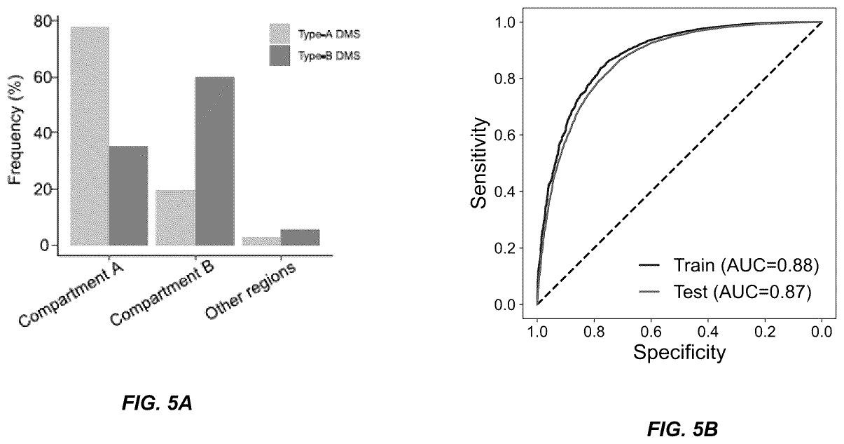

To further explore how the two types of differentially methylated CpG sites (DMSs) were associated with chromatin organizations of the genome, we overlapped the DMSs with compartment A or B that were enriched for open and closed chromatin, respectively. As shown in FIG. 5A, type-A DMSs (HCC-specific hypermethylated CpG sites) were about 4-fold more enriched in compartment A than in compartment B (78% vs 20%). In contrast, type-B DMSs (HCC-specific hypomethylated sites) showed a reverse trend of lower proportion in compartment A compared with B (35% vs 60%).

We split these hypomethylated and hypermethylated CpG sites into training and test sets and used cfDNA nucleosomal scores to predict methylation index for an individual CpG site using a CNN model. In various implementations, the CNN model can use two two-dimensional (2D)-convolutional layers, e.g., each having 16 filters with a kernel size of 4; other values may also be used. A batch normalization layer can be applied subsequently, followed by convolutional layers. The activation function of the rectified linear unit (ReLU) can be used for those convolutional layers; other activation functions can be used. A maximum pooling layer with a pool size of 2 was used; other values may also be used. A flattened layer can be further added, followed by a fully connected layer comprising 3200 neurons with the use of the ReLU activation function. The output layer with two neurons can be finally applied, with a softmax function to yield the hypermethylation probabilistic score for a CpG site. Other values can be used for any of the parameters above.

FIG. 5B shows a receiver operating characteristic curve (ROC) analysis for the prediction of methylation levels (e.g., indexes) of at sites in the training and test sets. We achieved an area (AUC) under the ROC curve of 0.87 in the test set. Thus, the nucleosomal signal patterns can be used to determine a methylation level at a particular site in a genome, including in a particular tissue type.

We checked the overlap between liver-specific CpGs (liver normal vs Buffy coat) and HCC-specific CpG (HCC tumor vs Buffy coat). The HCC-hypermethylated sites (118,544) and the liver-hypermethylated sites (258,630) had an overlap of 80543 sites. The HCC-hypomethylated sties (842,892) and the liver-hypomethylated sites (226,417) had an overlap of 78,755. Thus, tissue-specific sites or disease-specific sites for that tissue can be used to determine a methylation level in the particular tissue type.

Additionally or alternatively, the methylation alteration in a particular disease could be reflected by analyzing these nucleosomal signals associated with tissue-specific methylation patterns, as is evidence by the pattern difference for HCC tissue. The advantage of using the nucleosomal signals allowed for the removal of bisulfite treatment which would degrade DNA materials drastically. Such a technique is described in a later section.

### C. Method for Determining Methylation Level

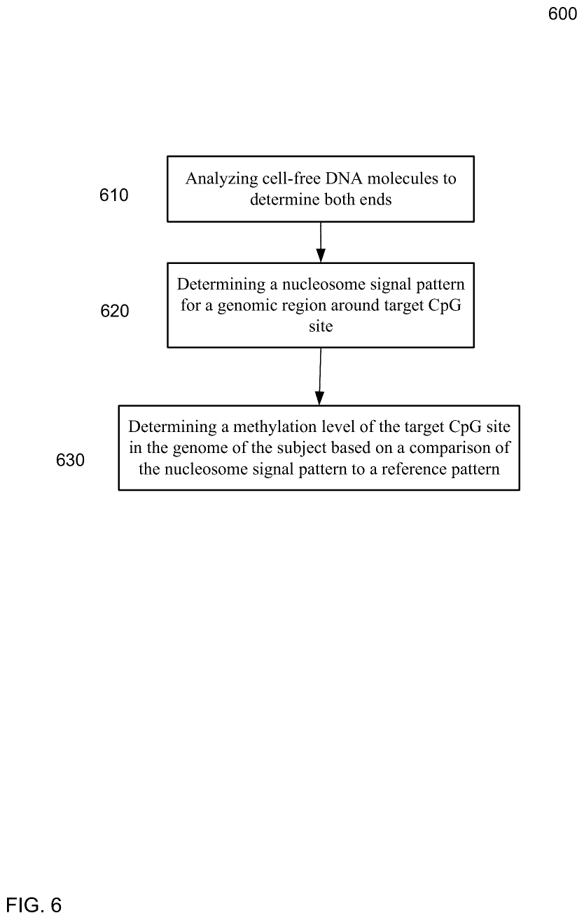

FIG. 6 is a flowchart illustrating a method 600 for determining a methylation level at a site using a nucleosome signal pattern according to embodiments of the present disclosure. Various examples of method 600 are described above. Method 600 can be performed partially or entirely using a computer system. Method 600 can determine the methylation level without having to use bisulfite conversion, which can degrade the DNA. Thus, a higher yield for the sequencing library can be obtained.

At block 610, a plurality of cell-free DNA molecules from a biological sample of the subject are analyzed. Various techniques can be used for such analysis in any of the methods described in the present disclosure. For example, the analysis can be performed using sequencing, such as massively parallel sequencing, targeted sequencing, and single molecule sequencing (e.g., using a nanopore or using real-time single molecule sequencing (e.g., from Pacific Biosciences)). The analysis can include the physical steps of performing such assays and receiving of the measurement data obtained from such assays or may just include receiving the measurement data.

Analyzing a cell-free DNA molecule can include determining a genomic position in a reference genome corresponding to at least one end of the cell-free DNA molecule. Thus, analyzing each of the plurality of cell-free DNA molecules can include determining two genomic positions in a reference genome corresponding to both ends of the cell-free DNA molecule. For example, one or more sequence reads of a DNA molecule (e.g., paired reads at the ends or a read for the entire molecule) can be aligned to the reference genome using any of various alignment techniques as will be appreciated by the skilled person. The alignment can be to some or all of the reference genome. The position determination can be to some or all of the reference genome, e.g., if only part of the genome is being analyzed. As examples, the amount of the genome analyzed can be greater than 0.01%, 0.1%, 1%, 5%, 10%, or 50% of the reference genome. Such an analysis may be performed for other methods described herein.

At block 620, a nucleosome signal pattern for a genomic region is determined around the target CpG site. The target CpG site can be at the center of the genomic region. The nucleosome signal pattern can be determined in various ways, as is described herein, e.g., using one or more normalization techniques. For example, the nucleosome signal pattern can include nucleosome scores, normalized nucleosome scores, or distribution-adjusted nucleosome scores (e.g., z-scores or t-scores). Such values are examples of a per-position nucleosome signal. The per-position nucleosome signal can be determined using cell-free DNA molecules that span a window around the genomic position and cell-free DNA molecules that end within the window around the genomic position.

The determination of the nucleosome signal pattern may be determined in the following manner. For each genomic position within the genomic region, a first amount of the plurality of cell-free DNA molecules that span a window around the genomic position can be determined. A second amount of the plurality of cell-free DNA molecules that end within the window around the genomic position can be determined. FIG. 2 provides an example description for such determinations. The per-position nucleosome signal can be determined using the first amount and the second amount, as described herein. For example, the first amount F and the second amount P can be determined using any of the following separation values: P/(F+P), P/(F−P), F/(F−P), P−F, F−P, F/P, or P/F.

Example sizes for the regions are provided herein. For example, the genomic region can be at least 140 bp in length. Other sizes for the genomic region are at least 100 bp, 110 bp, 120 bp, 130 bp, 150 bp, 160 bp, 170 bp, 180 bp, 190 bp, 200 bp, 250 bp, 300 bp, 400 bp, 500 bp, 600 bp, 700 bp, 800 bp, 900 bp, and 1,000 bp, as well as other sizes described herein.

As an example of normalization, the per-position nucleosome signal can be normalized using nucleosome signals from other genomic regions, e.g., flank regions. Such a normalization can use a mean value of the nucleosomal signals derived from the other genomic regions. The other genomic regions can be adjacent to the genomic region.

As another example for normalization, the nucleosome signal pattern is normalized using a region-level statistical value determined from one or more regions. Such a per-position nucleosome signal can be referred to as a nucleosome score. The one or more regions can include the genomic region or one or more other regions. The one or more regions can include one or more other regions that are adjacent to the genomic region. As examples, the region-level statistical value can be a mean or median value.

As another example for normalization, the per-position nucleosome signal can be normalized using a background signal that is dependent on a distance of the genomic position to the target CpG site, as is described in more detail in a later section. The background signal can be determined from one or more other genomic regions, each centered around a CpG site. The one or more regions can be selected randomly. The normalization can use a mean value or a median value of per-position nucleosomal signals derived from the other genomic regions respectively for each distance from the target CpG site.

As another example for normalization, the per-position nucleosome signal can be normalized using a distribution of reference nucleosome signals determined from one or more regions of one or more references samples. Normalizing using the distribution can includes, for each position, determining an aggregate statistical value and a dispersion value of the distribution of reference nucleosome signals and subtracting the aggregate statistical value from each per-position nucleosome signal and dividing by the dispersion value. For example, the distribution can be the normal distribution, where the aggregate statistical value is a mean of the reference nucleosome signals of the position and the dispersion value is a standard deviation.

At block 630, a methylation level of the target CpG site in the genome of the subject is determined based on a comparison of the nucleosome signal pattern to a reference pattern. The reference pattern can be determined from one or more training samples having a known methylation level. Examples of reference patterns are described herein. The comparison can be performed in various ways, e.g., using a machine learning model such as an SVM or other models described herein. The known methylation level can have various forms such as a range (e.g., of with 0.5%, 1%, 2%, 3%, 4%, 5%, 6%, 7%, 8%, 9%, or 10%) or be treated as specific values.

The methylation level can be that the target site is hypermethylated or hypomethylated. In another example, the methylation is a range for a methylation density at the target site. Examples of such a range can include 0-10%, 10-20%, 20-30%, 30-40%, 40-50%, 50-60%, 60-70%, 70-80%, 80-90%, and 90-100%. Other examples include smaller or wider widths for the ranges, such as 5%, 10%, 15%, 20%, or 25%.

The biological sample can be plasma or serum, as examples. The target site is known to be differentially methylated between a first tissue type and blood cells, and wherein the methylation level is determined for the target CpG site in the first tissue type. As examples, the first tissue type can be cancer tissue, fetal tissue, or the tissue of a particular organ.

### III. Cancer Classification Based on Nucleosome Signal Pattern

Nucleosomal signals from around sites (e.g., tissue-specific hypomethylation and/or hypermethylation sites) can used to detect and monitor pathologies (e.g., diseases such as cancers).

### A. Nucleosome Signal Patterns of Cancer Patients and Healthy Controls

We investigated the power of cancer detection by using cfDNA-based nucleosomal scores across CpG sites showing differential methylation patterns across various tissues. We determined the nucleosomal scores using cfDNA molecules originating from HCC-specific hypomethylated and hypermethylated CpG regions for healthy subjects and patients with cancer in Dataset A. Dataset A (Jiang et al. Cancer Discov. 2020; 10:664-673) includes paired-end sequencing data (75 bp×2, Illumina) of plasma cfDNA was obtained from healthy controls (n=38), subjects with chronic hepatitis B (HBV, n=17), patients with hepatocellular carcinoma (HCC, n=34), and pregnant women (n=30), respectively, with a median number of 38 million paired-end sequencing reads (range: 18-65 million).

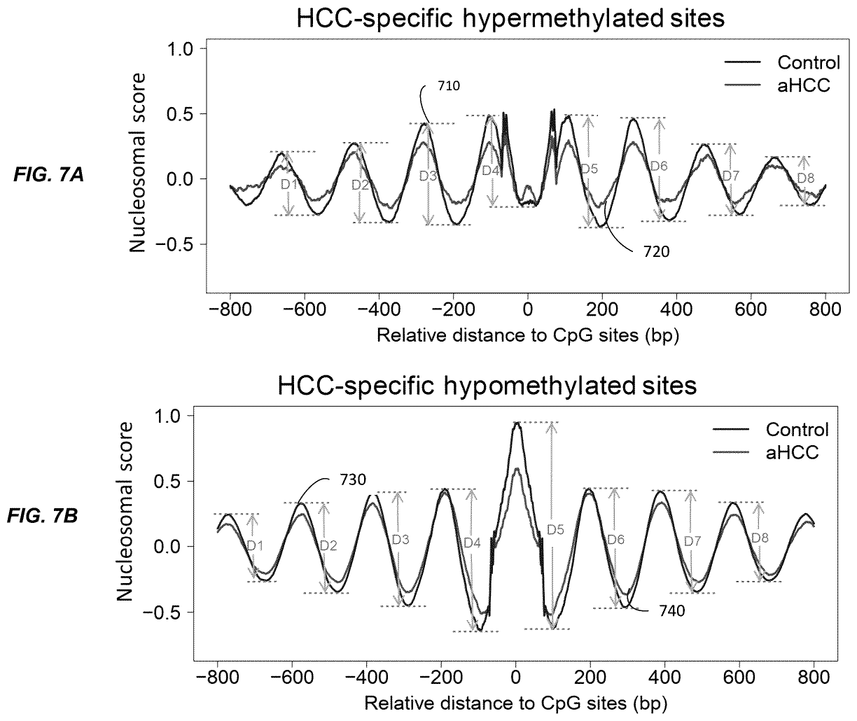

FIGS. 7A and 7B show mean nucleosome scores (normalized nucleosome signals) for HCC-specific hypermethylated and hypomethylated sites for healthy controls and patients with advanced-stage HCC (aHCC). Nucleosome signal pattern 710 and nucleosome signal pattern 730 are for healthy controls. Nucleosome signal pattern 720 and nucleosome signal pattern 740 are for HCC patients. A difference can be seen between the HCC patients and the healthy controls. Nucleosomal scores (−800 to 800 bp relative to a CpG site) surrounding HCC-specific hypomethylated and hypermethylated CpG sites are displayed.

For the HCC-specific hypermethylated sites, patients with advanced-stage HCC (aHCC) showed a weakened amplitude of nucleosomal patterns than healthy controls. This can be seen in the peak-trough distances of D1 to D8 shown in FIG. 7A.

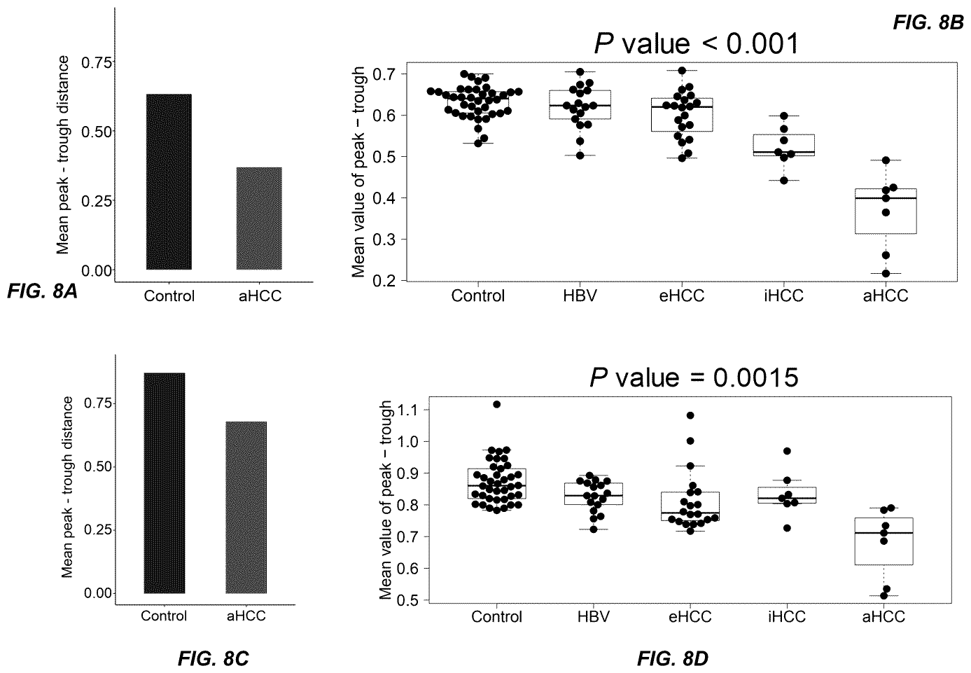

FIG. 8A shows the mean peak-trough distance for healthy controls and aHCC subjects. The bar values are averaged over samples, e.g., blue bar value is averaged over healthy samples and red bar value is averaged over aHCC samples. For example, the mean peak-trough distance can be determined across the peaks/troughs (e.g., D1-D8) for each sample, and then those means values can be averaged. The peak-trough pairs can be counted from the left or the right. The aHCC subjects showed a significant reduction in the distance. Such a use of the distance is one way that a sample nucleosome signal pattern (measured in a new sample) can be compared to each a reference pattern having a known classification, e.g., healthy or cancer.

FIG. 8B shows a mean peak-trough distance for various subjects: controls, HBV, early (eHCC), intermediate (iHCC), and advanced-stage HCC (aHCC). Each data point corresponds to a sample of a subject, with the mean peak-trough distance for a sample determined over the number of peak-trough pairs in the pattern. When comparing individual subjects, we found a significant reduction of nucleosomal amplitude (mean) in HCC patients compared to non-HCC subjects (P<0.001, Wilcoxon rank-sum test). Moreover, we observed a gradual decrease of nucleosomal amplitude over tumor stages, showing median amplitude of 0.62, 0.51, and 0.40 for early (eHCC), intermediate (iHCC), and advanced-stage HCC (aHCC), respectively, which further supports the contribution of tumor-derived DNA to weakening nucleosomal amplitude.

Similarly, FIGS. 8C and 8D show that nucleosomal amplitude was also decreased for HCC-specific hypomethylated CpG sites in HCC patients (P=0.0015, Wilcoxon rank-sum test), with aHCC patients showing the smallest peak-trough distance.

B. Normalizations Using Background and Mean of Healthy Samples (e.g., z-Score)

Normalization operations can be performed to make the differential signal more pronounced and robust. For display purposes, the differences of the hypermethylated and hypomethylated sites can be concatenated or viewed side-by-side, as shown below.

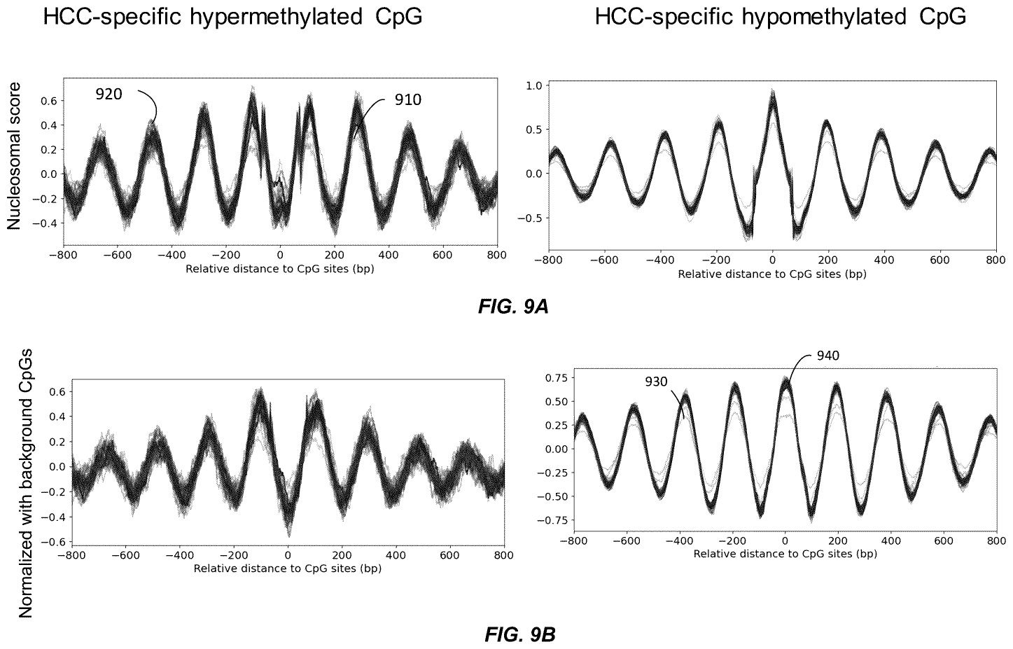

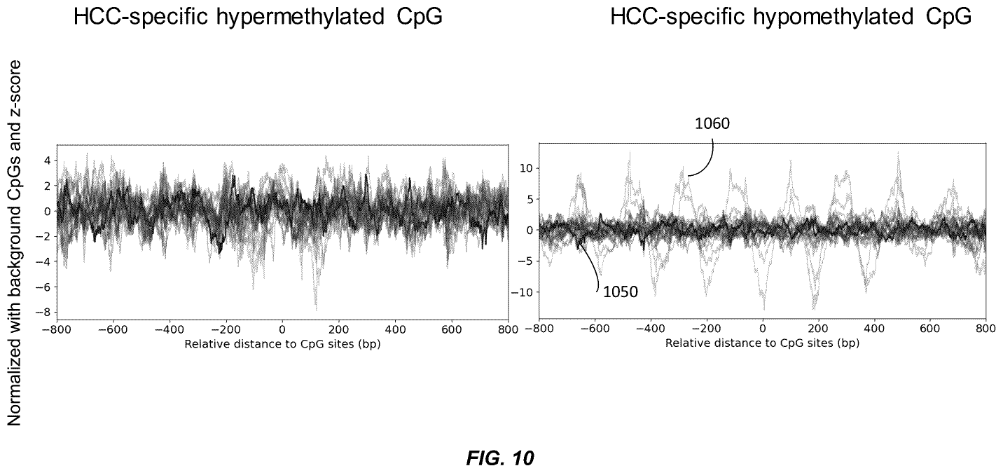

FIGS. 9A-9B and FIG. 10 show an example schematic diagram for normalizing nucleosomal score surrounding CpG sites. The nucleosomal scores from aggregated regions associated with hypomethylated and hypermethylated CpG sites were analyzed together (e.g., concatenated) to form a relatively large profile of nucleosomal scores. Each curve corresponds to a different sample. Each curve can be normalized using a background signal. A background signal can be determined in various ways. For example, the signal of a random CpG site or a set of multiple random CpG sites on the genome can be used to determine the background signal. Then a mean deviation normalization (e.g., z-score normalization) can be performed. Other distribution-adjusted nucleosome scores can also be used, besides a z-score.

FIG. 9A shows the signals for HCC-specific hypomethylated and hypermethylated sites. The curves 910 correspond to HCC samples. The curves 920 correspond to healthy samples. As mentioned above, the curves 910 with HCC have smaller amplitudes in peak and troughs (i.e., smaller distance) than the healthy samples.

The same HCC-specific sites from above are used. For a given curve of a sample, the nucleosomal scores can be determined by aggregating data across sites. One example can align 842,892 HCC-specific hypomethylated CpG sites at position “0” and then pool all DNA fragments mapped to −800 to 800 bp regions relative to “0” to construct one nucleosomal curve for the sample. Another example can determine an average of 842,892 nucleosomal curves at each position. The two techniques can be equivalent if each CpG site has sufficient DNA fragments.

FIG. 9B shows normalization with background nucleosomal score. The background nucleosomal score for each sample was obtained by aggregating 1,000,000 genomic regions, each centred on a CpG site. The HCC-specific nucleosomal scores were then normalized with the background nucleosomal scores. The curves 930 correspond to HCC samples. The curves 940 correspond to healthy samples. The smaller amplitudes of curves 930 with HCC than curves 940 with the healthy samples is more pronounced with the normalization.

We randomly selected 1,000,000 genomic regions each centered on a CpG site. Other numbers of random genomic regions can be used, e.g., at least 500, 1,000, 5,000, 10,000, 50,000, 100,000, 200,000, 500,000, and 2,000,000 regions. Sequenced cfDNA molecules from such genomic regions were aggregated together based on relative positions with reference to the central CpG site (i.e. position 0). The nucleosomal scores across those relative positions were determined as described herein and are referred to as background nucleosomal scores. As the number of background nucleosomal scores correspond to the size of the region, one can refer to a background pattern of the background nucleosomal scores.

The nucleosomal scores derived from the aggregated regions associated with hypomethylated and hypermethylated CpG sites were divided (or subtracted) by the background nucleosomal scores according to the relative positions. That is, each score is divided or subtracted by a respective value. The nucleosomal scores from aggregated regions associated with hypomethylated and hypermethylated CpG sites were concatenated to form a relatively large profile of nucleosomal scores.

FIG. 10 shows z-score normalization of nucleosomal scores. A set of healthy controls was used as a baseline to perform z-score normalization for each sample. Based on a set of healthy control subjects, we further determined the mean values (μ) and standard deviation values (δ) of normalized nucleosomal scores for each relative position. The normalized nucleosomal scores (s) in FIG. 9B (i.e., normalized based on the background pattern on a per-sample basis) were further translated into a z-score value based on the formula: (s−μ)/δ, which were referred to as nucleosomal z-scores. In other examples, a median or mode could be used instead of a mean or average. Thus, various aggregate statistical values can be used. Similarly, variance can be used instead of standard deviation. Thus, various measures for the spread in values (scores) can be used.

The z-score normalization can use the mean for the healthy samples and the standard deviation for the healthy samples. Thus, the formula can be (signal-mean_healthy)/(standard deviation_healthy). The z-score normalization can be done for each point separately. Thus, there is a different mean value and standard deviation for each point. Thus, the whole curve can be normalized point by point.

As shown in FIG. 10, HCC patient signal 1050 (normalized nucleosome z-score pattern) had unique patterns of nucleosomal z-scores with remarkable fluctuations that were deviated away from the healthy control signal 1060, which is relatively flat. Such unique patterns in patients with HCC suggested that the use of nucleosomal scores could inform patients with cancers. For the cell-free DNA from the cancer patients, we see the theoretical peaks here, which is the nucleosome signal pattern from the cancer. The cell-free DNA is a mixture of the cell-free DNA molecules from the blood cells and also from the tumor. But after normalization, the blood one is normalized so the HCC patient signal is from the tumor mainly.

The difference between the HCC patient signal 1050 and the healthy control signal 1060 is more pronounced in the HCC-specific hypomethylated versus the hypermethylated sites. Such difference might result due to about seven times as many hypomethylated sites being used as hypermethylated sites. Thus, an increase in the number of sites analyzed can increase accuracy. As examples, the number of sites can be 10, 20, 50, 100, 200, 500, 1,000, 2,000, 5,000, 10,000, 20,000, 50,000, 100,000, 500,000, one million, or more.

### C. Machine-Learning Techniques

The patterns of nucleosomal signals of various regions (e.g., raw nucleosome signal pattern or normalized using mean nucleosome signal (nucleosome score), background signals, and/or statistical values from other samples, such as z-score normalization) can be used for downstream classification analysis. Various machine learning models (e.g., as mentioned in the Terms section) can be used, such as but not limited to support vector machine (SVM), analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, Bayes classifier, maximum entropy classifier, conditional random field, nearest neighbor algorithm, probably approximately correct (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short-term memory, LSTM), hidden Markov model (HMM), linear discriminant analysis (LDA), k-means clustering, density-based spatial clustering of applications with noise (DBSCAN), random forest algorithm, etc. The normalized nucleosomal scores before z-score transformation, the nucleosomal scores before the correction based on background nucleosomal scores, or nucleosome signals before normalization based on other region(s) could be used as features for classification analysis.

The features for the ML model can be the nucleosome signals at a set of sites in the region. In various implementations, different nucleosome signal values could be used. The entire nucleosome signal pattern could be used but also a subset of signal values at only some of the positions in the region could be used. For example, every other position or a random selection; a reference pattern of a sample with a known classification would have the values for the same positions.

Different types of target sites can be used. For example, only hypomethylated sites or only hypermethylated sites can be used, or both sets can be used. The nucleosome values for a given class of sites (e.g., hypomethylated or hypermethylated) can be aggregated into a single pattern or the individual patterns can be used individually as features.

### 1. SVM Example Using HCC-Specific Differentially Methylated Sites

To illustrate the diagnostic power for the detection of HCC patients using nucleosome signal patterns (e.g., nucleosomal scores that may be background-adjusted or distribution-adjusted or raw nucleosome signals) of cfDNA molecules, we used an example ML model of a support vector machine (SVM) model to determine the probability of having HCC for a test sample. The input feature vector for a sample contained the nucleosomal score values at relative positions within 800-bp upstream and 800-bp downstream of the CpG of interest. Such CG sites included 842,892 HCC-specific hypomethylated and 118,544 hypermethylated CpG sites. Thus, a total of 3,202 features were used: the z-score at each position in the region.

The nucleosomal score values at the hypomethylated sites were aggregated, as was done for FIG. 9A. For example, for the 118,544 HCC-specific hypermethylated sites, all the cfDNA fragments mapped from those 118,544 regions (each region is 1601 bp long) were used to determine the nucleosomal signal value for position K (any position in the region) for the HCC-specific hypermethylation pattern. The same is done for the HCC-specific hypomethylated nucleosome signal pattern.

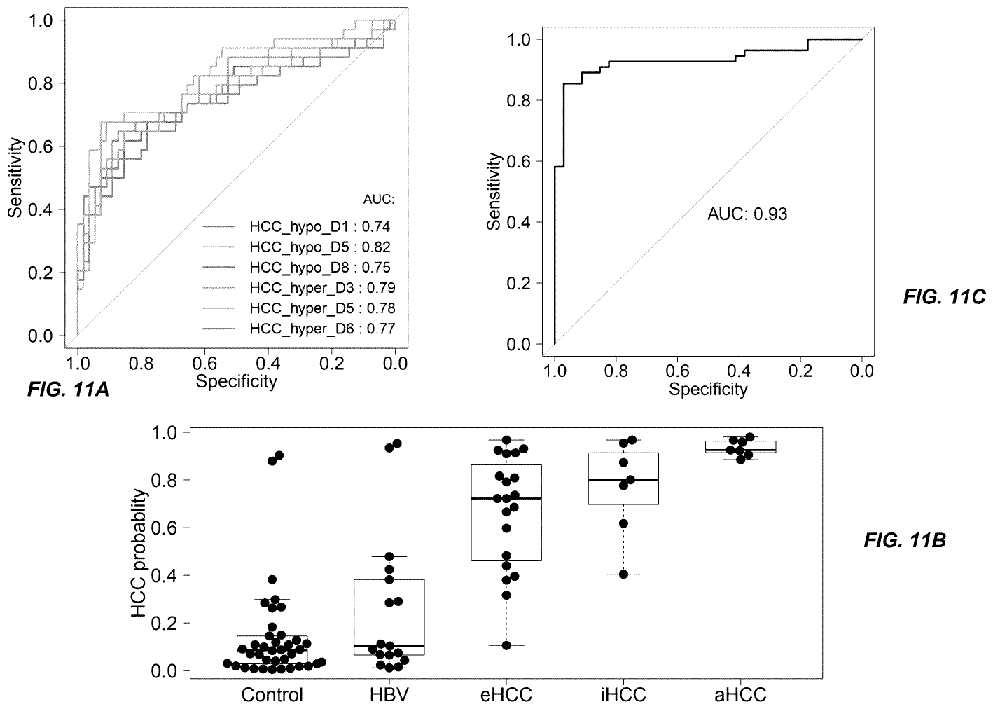

FIGS. 11A-11C show a set of graphs for assessing performance of using cfDNA nucleosomal signal patterns as a biomarker for cancer diagnostics.

FIG. 11A shows ROC curves for detecting HCC (i.e., differentiating between HCC and non-HCC) using various peak-trough pairs for HCC-hypermethylated sites and HCC-hypomethylated sites. As shown in FIG. 11A, directly using an individual peak-trough distance enables a classification of HCC and non-HCC subjects with a receiver operating characteristic (ROC) AUC of up to 0.82.

We further utilized the entire nucleosomal patterns (−800 to 800 bp regions shown in FIGS. 9A and B) as input feature for each sample, and a support vector machine (SVM) model was used to determine the probability of having HCC based on such patterns of nucleosomal scores.

FIG. 11B shows an HCC probability for different groups of subjects (known to have different classifications) using the SVM model. The HCC probability is determined from nucleosomal score, without background or z-score normalizations. The leave-one-out analysis showed that the probability of having HCC was significantly elevated in patients with HCC, compared with subjects without HCC (median: 0.81 vs 0.09; P<0.001, Wilcoxon rank-sum test). Additionally, patients with advanced HCC stages tended to have higher probabilities (median: 0.93) of having HCC compared with intermediate and early stages (median: 0.80 and 0.72 respectively). This can be due to higher fractional concentrations of tumor DNA for advanced stage. The determination of fractional concentrations of cell-free DNA from a particular tissue type are provided elsewhere in this disclosure.

FIG. 11C shows An ROC curve for the SVM model. The ROC analysis demonstrates that the approach based on nucleosomal patterns of cfDNA could differentiate between subjects with and without HCC, achieving an AUC of 0.93.

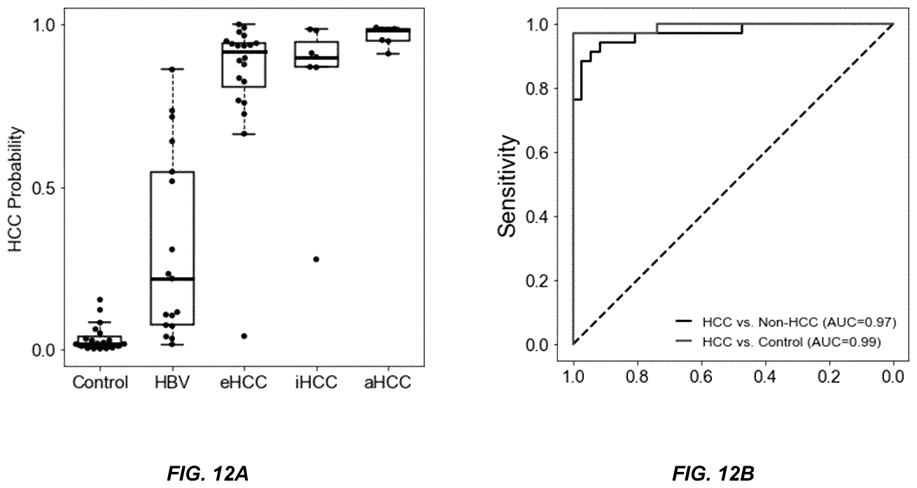

FIGS. 12A-12B show cancer detection using nucleosomal signals with alternative normalizations. Nucleosomal scores were obtained by dividing raw nucleosomal signals using the mean nucleosomal signal value of the flanking regions (−2000 to −1000 bp and 1000 to 2000 bp relative to a CpG site). Nucleosomal scores were further normalized by background CpG nucleosomal signal and z-score statistic, resulting in nucleosomal z-score.

FIG. 12A shows the HCC probability (vertical axis) based on a SVM model deduced by using nucleosomal z-score pattern (−800 to 800 bp relative to CpG site) surrounding HCC tissue-specific CpG sites. The HCC probability was determined using the SVM model described above. The probability of having HCC was significantly evaluated in patients with HCC, compared with subjects without HCC (median: 0.056 vs 0.934; P value: 1.2e-11, Wilcoxon rank-sum test). Additionally, patients with advanced HCC stages tended to have higher probabilities of having HCC compared with non-advanced stages (median: 0.98 vs 0.90). This can be due to higher fractional concentrations of tumor DNA for advanced stage. The determination of fractional concentrations of cell-free DNA from a particular tissue type are provided elsewhere in this disclosure.

FIG. 12B shows an ROC analysis using the predicted HCC probabilities to distinguish HCC and non-HCC subjects. The ROC analysis demonstrated that the approach based on nucleosomal z-score of cfDNA could differentiate between subjects with and without HCC, achieving an area under the curve (AUC) of 0.97.

In this study, we analyzed fragmentation patterns at the genomic regions with differential methylation statuses. The majority of the DMSs are not located in TSS (0.81%) and TFBS (25.5%) regions (i.e., 73.7% in other regions), suggesting that many other genomic regions harbor tissue-specific nucleosomal fragmentation patterns. Therefore, CpG-associated nucleosomal patterns in plasma DNA might contain molecular information independent from transcriptional activities and transcription factor binding events.

### D. Generalizability of Cancer Detection Model Across Different Datasets (Validation)

To examine the generalizability of nucleosomal pattern-based cancer diagnostics, we tested our approach on a separate cohort dataset even with different sequencing protocols or a different platform. We further hypothesized that the cancer detection model learned from one study cohort even with different sequencing platforms or protocols could be applied to other cohorts without re-training the model.

1. Validation with Same Sequencing Platform, but Different Protocols

To examine the generalizability of nucleosomal pattern-based cancer diagnostics, we tested our approach on a separate cohort Dataset B even with different sequencing protocols. We validate nucleosomal pattern-based cancer diagnostics in another cohort (Dataset B). Dataset B was published in 2015 (Jiang et al. Proc Natl Acad Sci USA. 2015; 112(11):E1317-1325). Dataset B (Jiang et al. Proc Natl Acad Sci USA. 2015; 112(11):E1317-1325) include paired-end sequencing data (75 bp×2, Illumina) of plasma cfDNA that was obtained from healthy controls (n=32), subjects with cirrhosis (n=36), subjects with HBV (n=67), and patients with HCC (n=90), respectively, with a median number of 31 million paired-end sequencing reads (range: 18-79 million).

The two datasets (A and B) used different experimental kits. Dataset A used DNA libraries prepared using TruSeq Nano DNA Library Prep Kit (Illumina). Dataset B used DNA libraries prepared by using the Kapa Library Preparation Kit (Kapa Biosystems).

### a) Trained and Tested on Dataset B

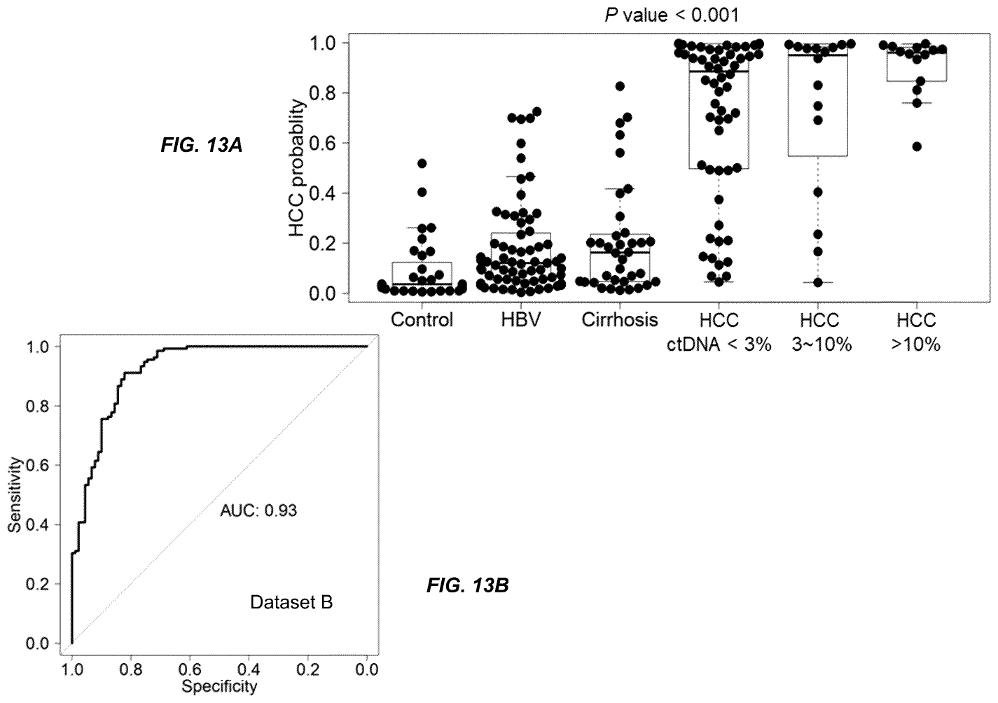

FIG. 13A provides the HCC probability for differentiating various levels of HCC patients from all non-HCC subjects for dataset B based on a leave-one-out analysis using SVM. FIG. 13B shows an ROC curve for differentiating HCC patients from all non-HCC subjects. Nucleosomal scores were used for the nucleosome signal patterns. Nucleosomal scores were used, without background and z-score normalizations.

As shown, we could achieve a great separation between HCC patients and non-HCC subjects (including healthy controls, patients with cirrhosis, and HBV carriers) using the probability of having HCC (median: 0.93 vs 0.10; P<0.001, Wilcoxon rank-sum test; FIG. 13A), with a ROC AUC of 0.93 for differentiating HCC patients from all non-HCC subjects (FIG. 13B). Furthermore, we found a coinciding increase in predicted HCC probability with tumor burden (median: 0.96, 0.95, and 0.89 for patients with below 3%, 3˜10%, and over 10% circulating tumor DNA(ctDNA) fraction, respectively FIG. 13A). The ctDNA fractions were estimated based on copy number aberrations according to ichorCNA (Adalsteinsson et al. Nat Commun. 2017; 8:1324). If one focused on patients with cirrhosis without HCC, the AUC could be still as high as 0.91 to distinguish HCC and cirrhosis patients. As cirrhosis is the most important risk factor of developing HCC, the ability of this approach to accurately differentiate patients with HCC and cirrhosis is clinically important.

### b) Trained on Dataset B and Tested on Dataset A

We further challenged the robustness of this analytic framework by testing whether the cancer detection model trained from one study cohort could be generalized to another cohort even with different sequencing protocols. To this end, we attempted to differentiate between patients with and without HCC in Dataset A, by making use of the SVM model for cancer detection that was trained based on Dataset B. Nucleosomal scores were normalized with background CpG nucleosomal signal and z-score statistic in each dataset to minimize inter-dataset bias.

In the analysis of Dataset B, we used 32 healthy subjects, 36 subjects with cirrhosis, and 67 HBV carriers, and 90 patients with HCC. We used 16 of the healthy controls to deduce the mean values and standard deviations of nucleosomal scores across positions relative to the CpG sites of interest. These mean values and standard deviations were used to determine the nucleosomal z-scores in independent testing samples in Dataset B. Similarly in Dataset A, a subset of healthy controls (n=19) was used to deduce the mean values and standard deviations to further determine the nucleosomal z-scores of the remaining samples in Dataset A. We performed normalizations for Dataset A and B separately.

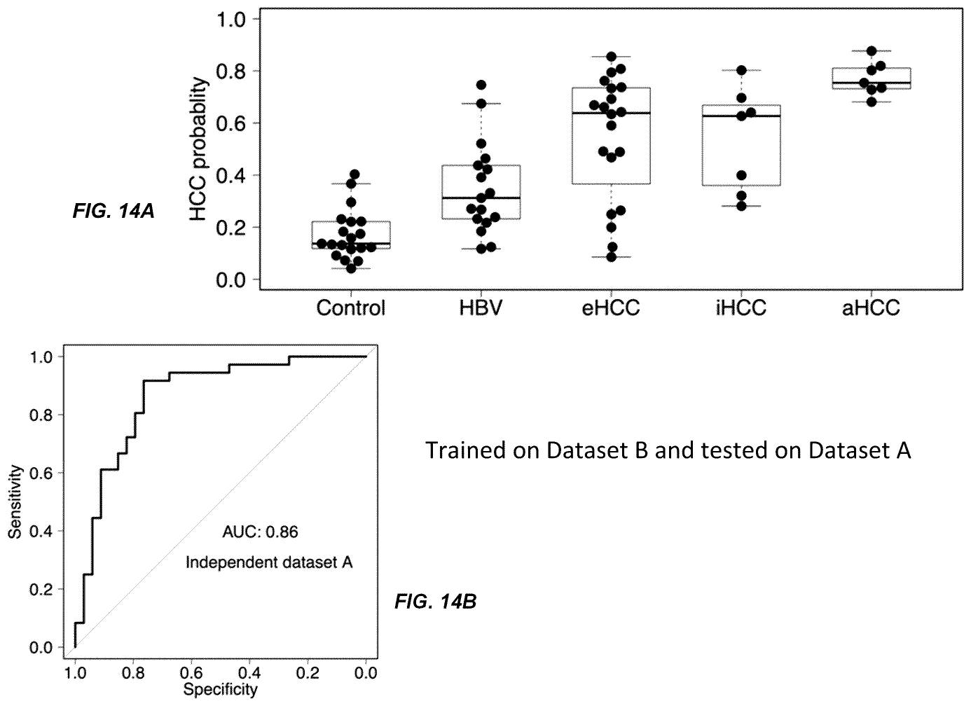

FIG. 14A provides the HCC probability of the SVM model trained with a dataset using one sequencing protocol for differentiating various levels of HCC patients from all non-HCC subjects for a dataset using a different sequencing protocol. The SVM model trained from Dataset B was applied to predict on independent Dataset A. FIG. 14B shows an ROC curve for differentiating HCC patients from all non-HCC subjects in the test Dataset A. Normalized z-scores were used for the nucleosome signal patterns.

As shown, we could observe significantly higher probabilities of having HCC in patients with HCC, compared with non-HCC subjects in the independent test dataset (P<0.001, Wilcoxon rank-sum test; FIG. 14A). We could achieve an AUC of 0.93 for differentiating HCC patients from healthy controls and an AUC of 0.86 for differentiating from all non-HCC subjects in which HBV carriers were also included (FIG. 14B), demonstrating that the nucleosomal pattern-based approach for cancer detection could be well generalized across different datasets.

### c) Trained on Dataset a and Tested on Dataset B

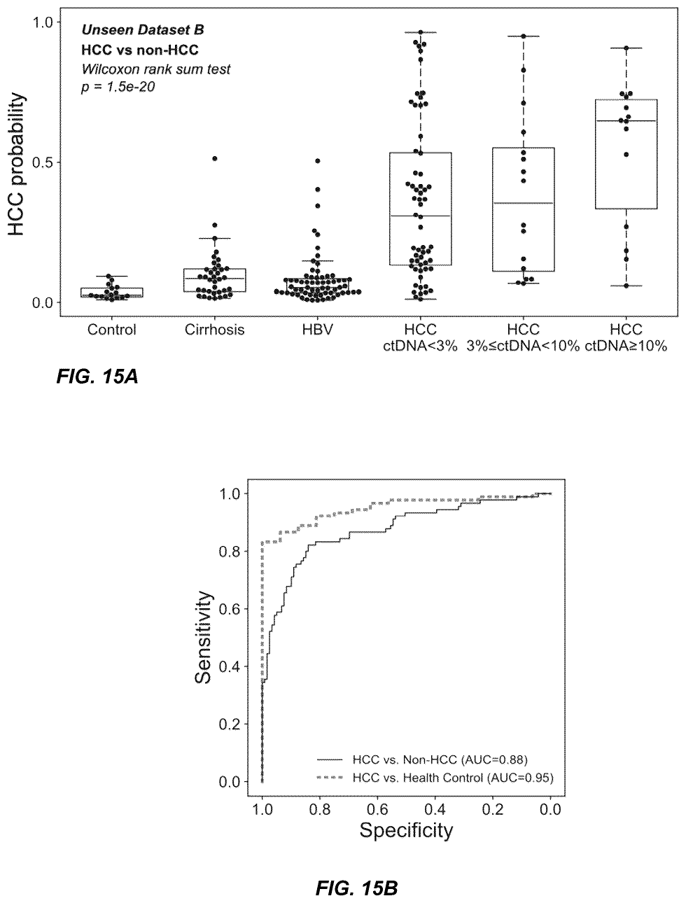

FIGS. 15A-15B shows cancer detection shows (HCC probability) in Dataset B based on a SVM model trained from Dataset A using nucleosomal signals. In this example, raw nucleosomal signal was normalized to nucleosomal score by dividing the mean signal of the flanking regions (−2000 to −1000 bp and 1000 to 2000 bp relative to a CpG site), followed by normalizations of background CpG nucleosomal signal and z-score statistic.

In FIG. 15A, the groups are healthy controls, cirrhosis, HBV, and HCC. We could achieve a great separation between non-HCC subjects and HCC patients, using the probability of having HCC deduced by the model trained from Dataset A (P=1.5e-20, Wilcoxon rank-sum test). Furthermore, we found an increasing trend in the probability of having HCC for those patients with higher tumor DNA fractions.

FIG. 15B shows ROC analysis of predicted HCC probabilities in distinguishing HCC and non-HCC subjects from Dataset B. The ROC curve revealed an AUC of 0.95 for differentiating HCC patients from healthy controls and an AUC of 0.88 for differentiating from all non-HCC subjects.

In summary, FIGS. 13A-13B show cancer detection using leave-one-out analysis in dataset B, which shows embodiments work in another cohort. FIGS. 14A-14B show cancer detection in dataset A, using a SVM model trained from dataset B, which shows embodiments enable having one model generalized across different datasets. FIGS. 15A-15B shows cancer detection in dataset B, using a SVM model trained from Dataset A.

2. Survival Rates of HBV Subjects with High and Low HCC Risk

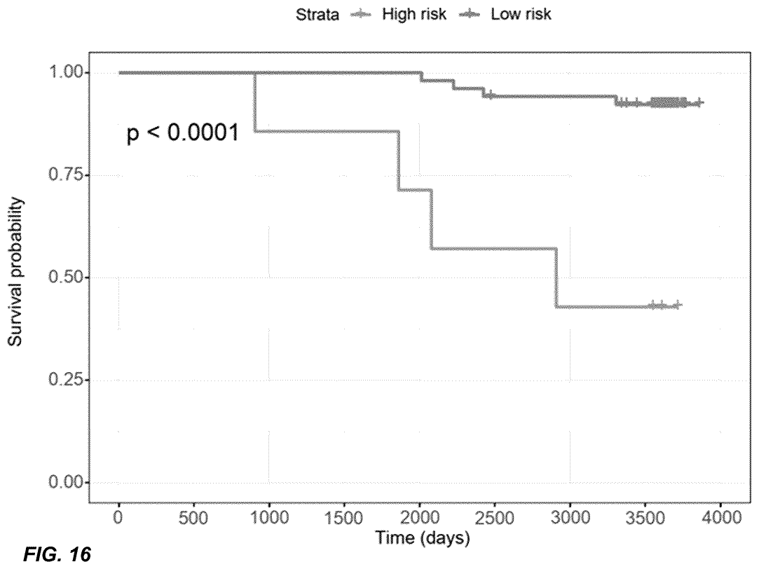

FIG. 16 shows a graph that identifies Kaplan-Meier analysis of survival in HBV subjects stratified in two risk groups based on predicted HCC probability shown in FIG. 15A. HBV subjects with high risks of HCC (HCC probability>0.12) had significantly worse survival compared to those with low risks (HCC probability<0.12).

Importantly, a follow-up clinical information analysis of Dataset B revealed that among 7 HBV carriers predicted to be as high risk of HCC (HCC probabilities>0.12), 4 HBV carriers (57.1%) had developed HCC within 5 years after the blood test; whereas among 52 HBV carriers with low risk of HCC (HCC probabilities<0.12), only 6 HBV carriers (11.5%) had developed HCC. The odds ratio for HCC development between these two groups was almost 5. The Kaplan-Meier analysis of survival showed that HBV subjects with a high risk of HCC had significantly worse survival rates compared to those with the low risk of HCC (P value<0.0001).

The results suggested that one could use the probability of having HCC deduced from nucleosomal signals to perform patient stratification, for example, sorting out the subgroup with high risks of HCC from the HBV carriers. Those patients with high risks of HCC would be recommended to adopt more frequent clinical surveillance. These data also suggested that the approach presented in this disclosure was more sensitive to screen out HBV carriers who may ultimately develop HCC within in a certain time range, compared to the conventional clinical surveillance and management such as abdominal imaging (ultrasound, CT, or MRI).

Accordingly, the subject can be determined to have a viral infection, e.g., by detecting viral DNA fragments, viral proteins, or other mechanisms. The classification of the level of the cancer can be that cancer does not exist but that the subject is at higher risk than other subjects having the viral infection.

More generally, the classification of the level of the cancer can be a likelihood of the subject getting cancer in future. When the likelihood exceeds a threshold (determined by comparing), The subject can be monitored based on the likelihood exceeds the threshold. The monitoring would be at a higher rate than tan if the subject had a likelihood that was below the threshold. Examples of monitoring (also referred to as screening modalities) are provided elsewhere herein.

3. Validation with Difference Sequencing Platform

In another example, we validated our approach of cancer detection in cfDNA data with different sequencing platform. Dataset C (Yu et al. Clin Chem. 2023; 69(2):168-179) includes single-molecule sequencing data (Oxford Nanopore) of plasma cfDNA from subjects with HBV (n=6), and patients with HCC (n=8), respectively, with a median number of 9 million paired-end sequencing reads (range: 1.6-31 million).

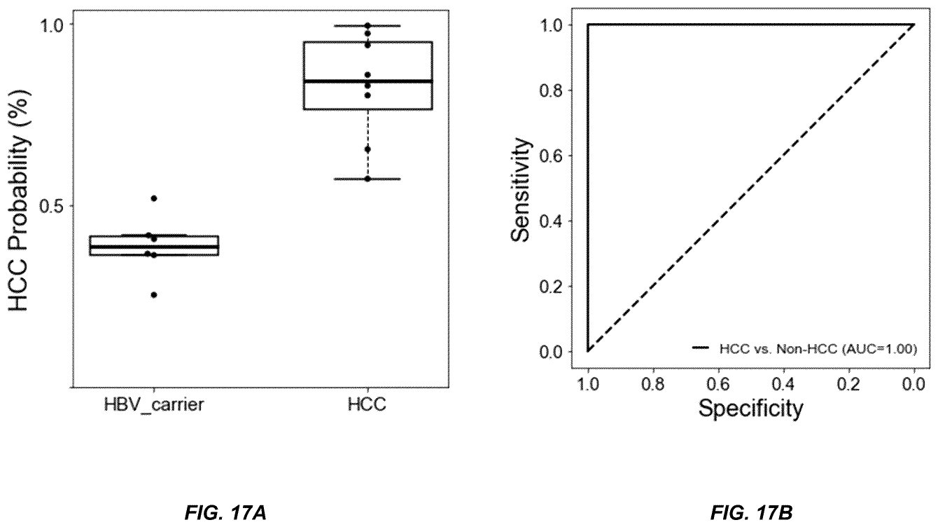

FIGS. 17A-17B show a set of graphs for evaluating performance of nucleosomal scores for cancer diagnosis in Dataset C generated using nanopore sequencing. FIG. 17A shows HCC probability predicted for the samples from Dataset C based on a leave-one-out analysis using SVM. As shown in FIG. 17A, nucleosomal score is still valid for differentiating HCC from non-HCC (P=0.0019, Wilcoxon rank-sum test) even though Dataset C was prepared by nanopore sequencing technology (e.g. Oxford Nanopore Technologies). FIG. 17B shows ROC analysis of predicted HCC probabilities in distinguishing HCC and non-HCC subjects from Dataset C, in which the AUC was 1.0. The analysis is limited by the cohort size because there are only six HBV and eight HCC patients. But we can see the promise of the method and the biomarker.

4. Accuracy with Lower Number of Fragments

Downsampling analysis was performed for dataset A to determine the minimal number of fragments required for a robust classification between subjects with and without HCC. As a result, relative to the use of all fragments (median: 38 million), the use of 5 million fragments could achieve a comparable accuracy of classification (AUC: 0.93 vs 0.92). The values of the number of fragments and AUC are: All (median: 38M) with AUC of 0.931; 10 million fragments with AUC of 0.9289; 5 million fragments with AUC of 0.916; 2 million fragments with AUC of 0.9091; 1 million fragments with AUC of 0.8861; 0.5 million fragments with AUC of 0.7631, 0.2 million fragments with AUC of 0.7471; and 0.1 million fragments with AUC of 0.7283.

### E. Performance of Cancer Classification for Different Settings

Different settings can be used, e.g., for normalization or for what constitutes a hypermethylated or hypomethylated site.

### 1. Normalizations

The generalizability of nucleosomal signals across different sequencing protocols might be attributed to the data normalization steps used in this disclosure. To further demonstrate the effect of data normalization on classification analysis, we determined the AUC value corresponding to each normalization step.

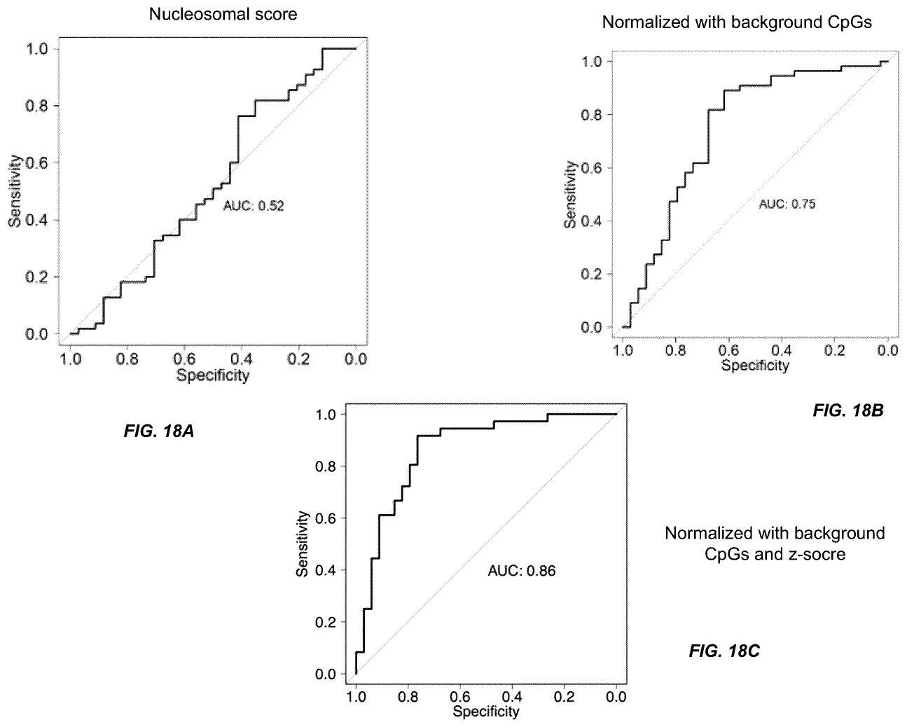

FIGS. 18A-18C show a set of ROC curves for evaluating the effect of data normalization for nucleosomal score patterns and cancer detection. The SVM model trained from Dataset B was tested on Dataset A. We used the model trained from Dataset B to analyze the sample in Dataset A, to evaluate the effect of data normalization on cancer diagnosis based on ROC analyses.

FIG. 18A shows analysis of cancer detection using nucleosomal score. The AUC in differentiating subjects with and without HCC was only 0.52 in Dataset A if using a model trained from Dataset B.

FIG. 18B shows analysis of cancer detection using nucleosomal score normalized with background CpG nucleosomal score. The AUC was rapidly elevated to 0.75 for differentiating HCC from non-HCC subjects.

FIG. 18C shows analysis of cancer detection using nucleosomal score normalized with both background CpG nucleosomal score and z-score statistic, called nucleosomal z-score. The AUC was further increased to 0.86 for differentiating HCC from non-HCC subjects. These data suggested that the normalization using a distribution of healthy samples improved accuracy significantly for the model generalization across platforms.

### 2. Different Methylation Cut-Offs to Define DMSs

As described above, hypermethylated sites and hypomethylated sites can be defined by a percentage for the methylation density at that site relative to other tissue types or other sites. Hypermethylated sites and hypomethylated sites can be defined for a particular tissue type or more generally across all tissue types. HCC-specific hypermethylated sites and HCC-hypomethylated sites were used for Dataset A. Different cutoffs were tested and shown to have an effect on the result.

We conducted analyses using two sets of cut-offs of <20% methylation in buffy coat and >80% in tumor (tumor-hypermethylated sites) and vice versa (tumor-hypomethylated sites, i.e. <20% methylation in tumor and >80% in buffy coat), and cut-offs of <10% methylation in buffy coat and >90% in tumor and vice versa.

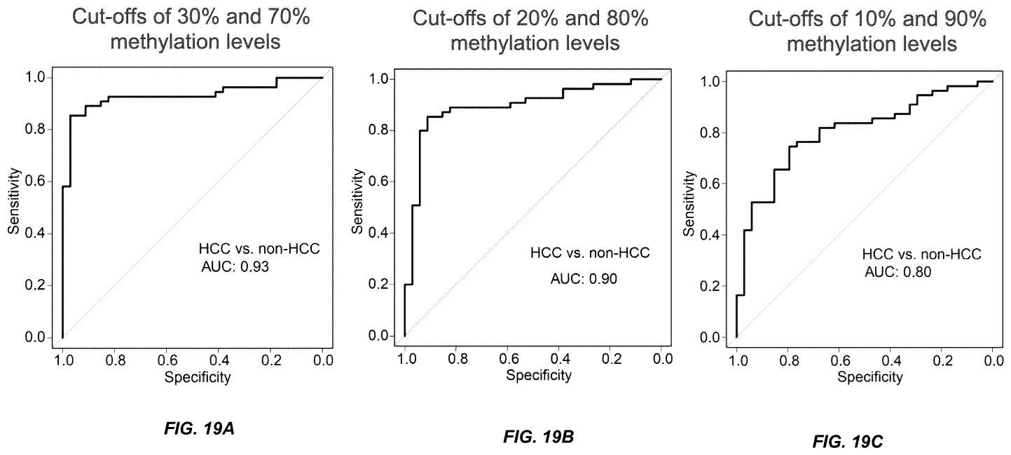

FIGS. 19A-19C show ROC curves of HCC detection using nucleosome signal patterns based on differentially methylated sites (DMSs) defined by different criteria of methylation levels between the buffy coat and tumor tissues. The methylation cut-offs of 30% and 70% gave rise to an AUC of 0.93 for detecting cancer in Dataset A. When using methylation cut-offs with more stringent criteria, the AUC values for cancer detection are 0.90 (<20% in buffy coat and >80% in the tumor, and <20% in the tumor and >80% in buffy coat) and 0.80 (<10% in buffy coat and >90% in the tumor, and <10% in the tumor and >90% in buffy coat).

### F. Results Using Random CpG Sites

In contrast to the results above, no such accurate prediction was seen if one employed the nucleosomal scores of cfDNA on the basis of the CpG sites, which do not show differential methylation patterns across tissues (e.g. the difference of methylation density between buffy coat and HCC tumor was less than 5%).

We tested to using random CpG sites for cancer detection in Dataset A when using a model trained from Dataset B.

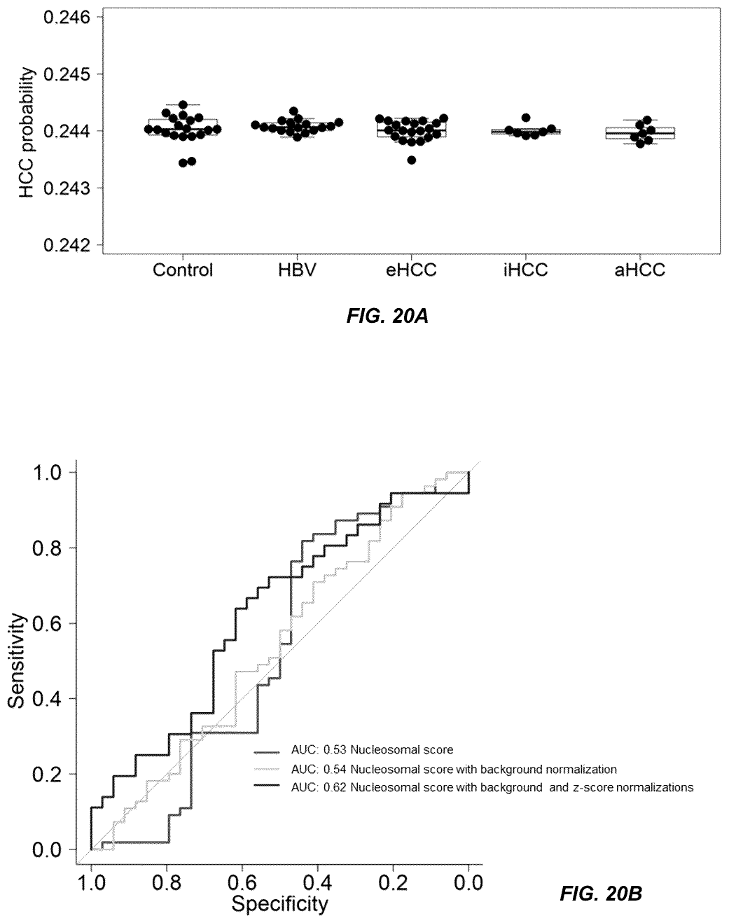

FIGS. 20A-20B shows a set of graphs that assess performance of nucleosomal score analysis for random CpG sites. Median subtraction is used for the normalization. FIG. 20A shows HCC probability deduced by using nucleosomal z-score surrounding random CpG sites. As shown in FIG. 20A, there was no observable significant difference in the nucleosomal score between subjects with and without HCC (P value: 0.08, Wilcoxon rank-sum test), with an AUC of 0.62.

FIG. 20B shows performance of cancer detection using nucleosomal score analysis for random CpG sites. The ROC analysis uses predicted HCC probabilities to distinguish HCC and non-HCC subjects. The model using nucleosomal scores based on random CpG sites could not detect cancer, no matter without normalization (AUC=0.53), with background CpG normalization (AUC=0.54), or with both background CpG and z-score normalizations (AUC=0.62).

This data shows that using both the fragmentation of the nucleosome signal patterns and the methylation information (i.e., using the signal patterns at differentially methylated sites for the cancer) provide increased accuracy.

G. Comparison with Other Techniques

We compared the use of nucleosome signal patterns to detect cancer with other methods. Based on the data in Dataset A (Jiang et al., 2020), we evaluated three alternative approaches, namely, ichorCNA, the ratio of short fragments, and end motif patterns. IchorCNA is based on copy number aberrations. It has been reported that cfDNA fragments from cancer patients tend to be shorter and exhibit a greater diversity of end motifs. The ratio of fragments below 150 bp and motif diversity score (MDS) were reported to be informative for cancer detection (Jiang et al., 2020).

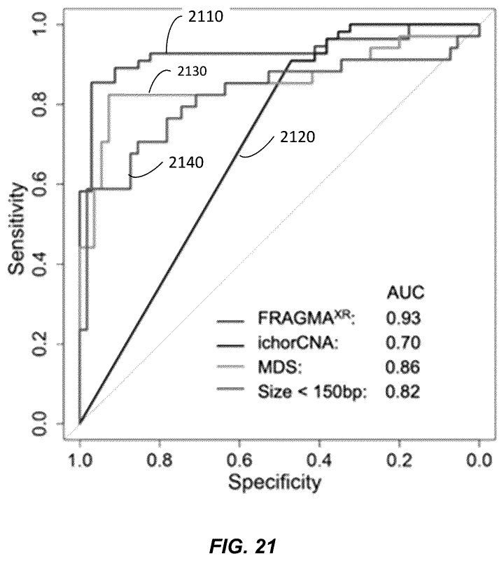

FIG. 21 shows performance of nucleosome signal patterns (FRAGMA^{XR}) relative to other techniques for Dataset A. As shown, we found that FRAGMAXR 2210 (AUC=0.93) appeared to be superior to these methods (AUCs: 0.70 for ichorCNA 2120, 0.82 for the short fragment ratio 2140, and 0.86 for MDS 2130).

### H. Other Cancer Types

To show that such use of nucleosome signal patterns is applicable to other cancer types, we further conducted additional analyses by applying nucleosome signal patterns to the detection of different types of cancers, including lung, ovarian, and breast. To this end, we determined the differentially methylated sites (DMSs) for these cancer types, by comparing methylation levels between white blood cells and tumor tissues based on bisulfite sequencing data generated by NovaSeq 6000 (Illumina).

We employed the same criteria for defining the two categories of DMSs. In brief, for type-A DMSs (tumor-hypermethylated CpG sites), the methylation level of a CpG in the buffy coat is required to be <30%, while the corresponding level in the tumor tissue of interest is required to be >70%. For type-B DMSs (tumor-hypomethylated sites), the opposite requirement is applied. As a result, we identified 45,848 type-A and 117,414 type-B DMSs for lung cancer; 133,123 type-A and 348,608 type-B DMSs for ovarian cancer; 254,669 type-A and 588,133 type-B DMSs for breast cancer.

We further constructed and analyzed the nucleosomal patterns around DMSs for the publicly available cfDNA sequencing data from a previous study (Cristiano et al., Nature 570, 385-389, 2019), consisting of lung (n=12), breast (n=54), and ovarian (n=28) cancers, as well as from healthy individuals (n=245).

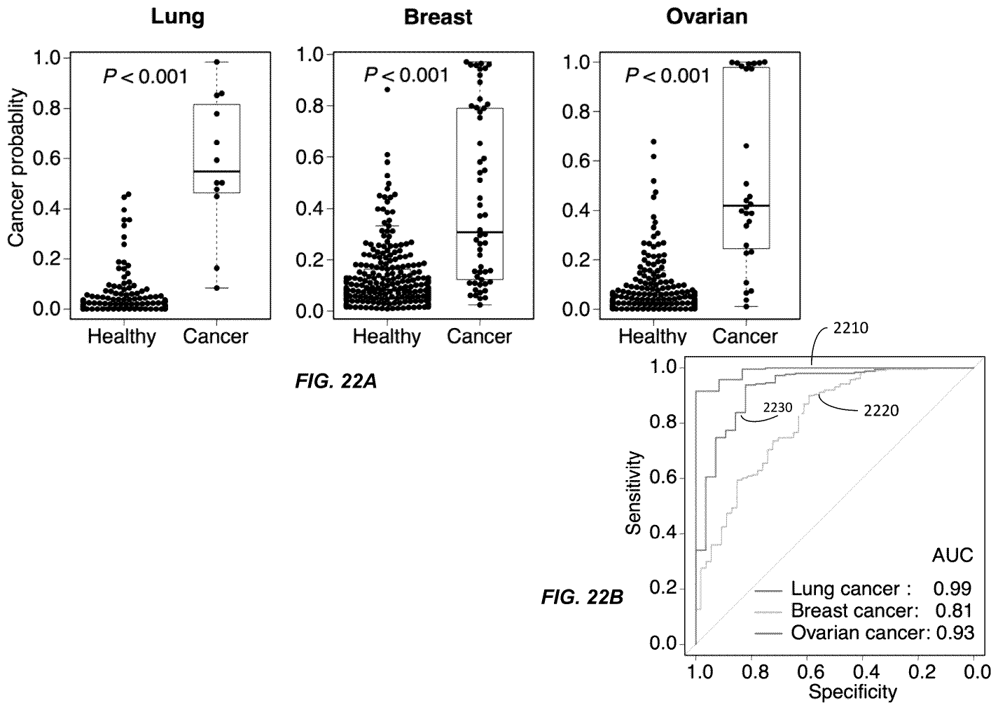

FIGS. 22A and 22B show cancer detection results for lung, breast, and ovarian cancers using nucleosome signal patterns. FIG. 22A shows probabilistic scores of having cancer in datasets with different cancer types. FIG. 22B shows a ROC analysis using predicted probabilistic scores of having cancer. Making use of these nucleosomal patterns, we demonstrated AUC values of 0.99, 0.81, and 0.93 for lung 2210, breast 2220, and ovarian cancers 2230, respectively.

We also analyzed the effect of increased tumor fraction on the probabilities for the different cancers. A higher probabilistic score of having cancer was generally observed in those patients with higher tumor DNA fraction across all three cancer types.

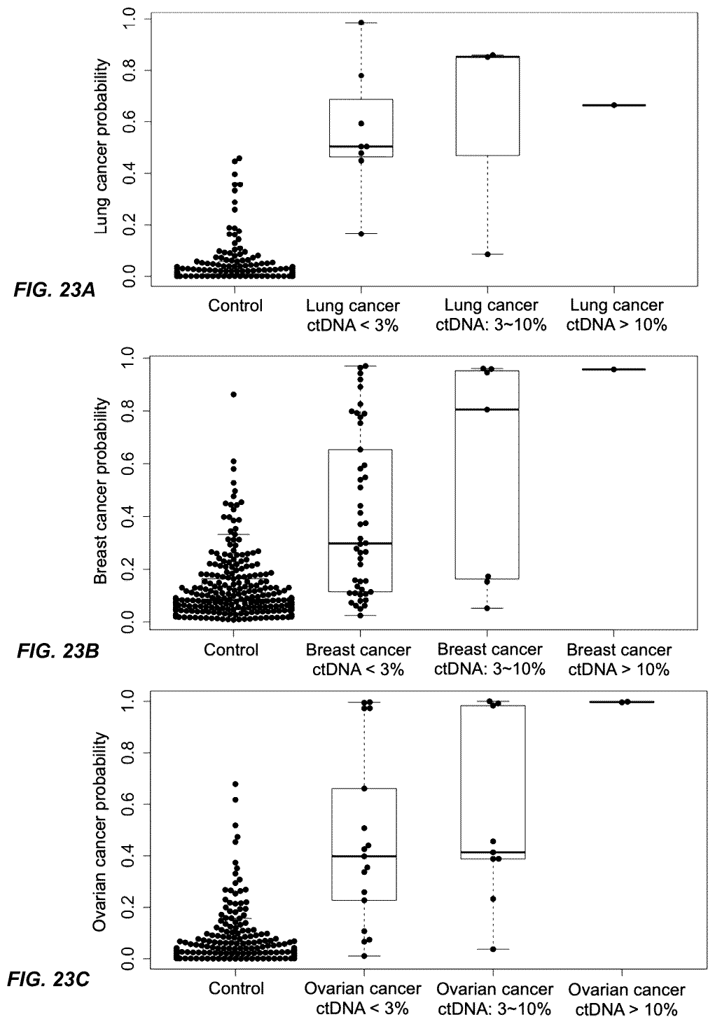

FIGS. 23A-23C show probabilistic scores of having cancer predicted depending on tumor DNA fraction in plasma of patients with cancers. Cancer patients were divided into three groups based on the tumor DNA fraction estimated using ichorCNA that was based on copy number aberrations.

These data indeed suggested that embodiments of the present disclosure are applicable to multi-cancer detection.

To test whether embodiments can distinguish different cancer types, we trained a model for multi-cancer classification using the plasma samples from cancer patients, which included liver (n=34), lung (n=12), breast (n=54), and ovarian (n=28) cancers. To this end, using nucleosomal z-scores around DMSs as input features, we employed a support vector machine (SVM)-based multiclass classification model to classify different cancer types. Leave-one-out analysis showed that this SVM model could correctly identify 79% (27/34) of liver cancer, 83% (10/12) of lung cancer, 70% (38/54) of breast cancer, and 71% (20/28) of ovarian cancer samples. Other model types can be used as well, such as neural networks, decisions trees, and other model types described herein. Such data shows that embodiments can differentiate among different cancers.

### I. Method for Cancer Detection

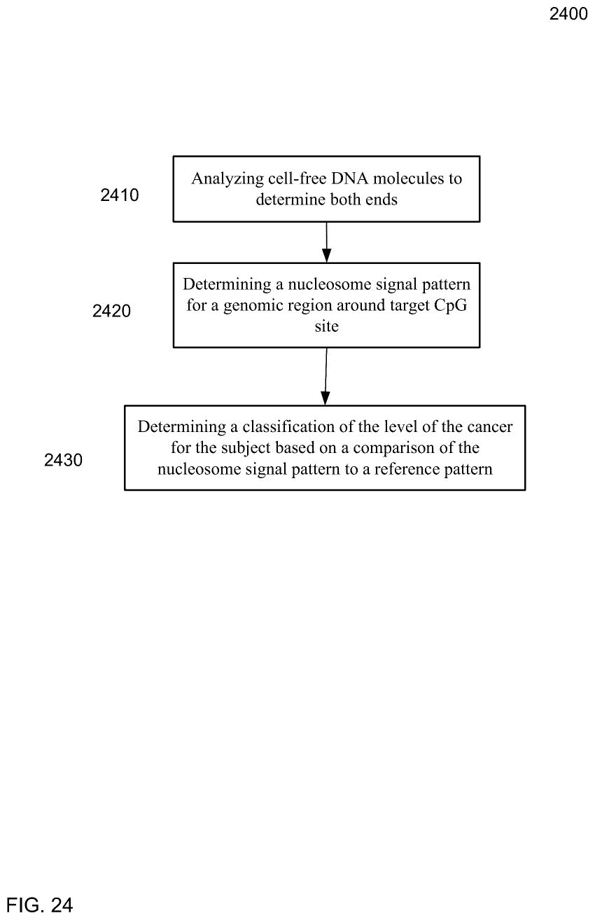

FIG. 24 is a flowchart illustrating a method 2400 analyzing a biological sample to determine a level of a cancer in the biological sample of a subject. Various examples of method 2000 are described above. Method 2400 can be performed partially or entirely using a computer system.

At block 2410, a plurality of cell-free DNA molecules from a biological sample of the subject are analyzed. Block 2410 can be performed in a similar manner as block 610 of method 600.

At block 2420, a nucleosome signal pattern for a genomic region is determined around the target CpG site. Block 2420 can be performed in a similar manner as block 620 of method 600. The target CpG site can be differentially methylated in a target tissue type (e.g., of an organ) relative to one or more other tissue types, e.g., as described above. As examples, the biological sample can be plasma or serum, and the one or more other tissue types include blood cells.

In some embodiments, the nucleosome signal pattern can be determined by aggregating over a plurality of genomic regions around a plurality of CpG sites. The plurality of CpG sites can be hypermethylated relative to the one or more other tissue types. The plurality of CpG sites can be hypomethylated relative to the one or more other tissue types. In other examples, both can be used with separate patterns for each.

At block 2430, a classification of the level of the cancer for the subject is determined based on a comparison of the nucleosome signal pattern to a reference pattern. Block 2030 can be performed in a similar manner as block 630 of method 600.

The reference pattern can be determined from one or more training samples having a known level of the cancer. The level of cancer can be determined for the target tissue type. For example, for selected CpG sites differentially methylated in blood and liver tumor, the cancer classification can detect liver cancer. Similarly, for CpG sites differentially methylated in blood and a colon tumor, then the model can detect colon cancer. Other example tissue types (e.g., lung, breast, etc.) are provided herein. Using all of such patterns (which can include a single concatenated pattern), embodiments can provide a pan-cancer test. Such tissue types used for determining the differentially methylated sites can be healthy or cancerous tissue.

Examples of reference patterns are described herein. The comparison can be performed in various ways, e.g., using a machine learning model such as an SVM or other models described herein. Accordingly, determining the classification of the level of the cancer can include inputting the nucleosome signal pattern into a machine learning model that was trained using the reference pattern of the one or more training samples. As another example, the comparison of the nucleosome signal pattern to the reference pattern can use a peak-to-trough distance, which can be a threshold value that represents the reference pattern.

For at least one of the one or more training samples, the known level of the cancer can be that the subject does not have the cancer.

In some embodiments, the subject can have a viral infection, and the classification of the level of the cancer can be that cancer does not exist but that the subject is at a higher risk for cancer than other subjects having the viral infection. For example, FIG. 16 shows such an instance for HBV infection.

The classification of the level of the cancer can be a likelihood (e.g., a probability) of the subject getting cancer in future. In such instances, the likelihood can be compared to a threshold. Based on the likelihood exceeding the threshold, monitoring of the subject can be performed. For example, the subject can be monitored by performing screening at a higher rate when the likelihood exceeds the threshold than is performed when the likelihood is less than the threshold. Examples of monitoring are provided elsewhere, e.g., in a treatments and monitoring section.

One or more advantages of using nucleosome signal patterns are that bisulfite sequencing is not needed, so one can perform methylation-aware fragmentomic signal from non-bisulfite sequencing data of cell-free DNA molecules, according to some embodiments.

IV. Determining Fraction of DNA from Particular Tissue Type

A fractional concentration of cell-free DNA from a particular tissue type can also be determined using nucleosome signal patterns. For example, the nucleosome signal patterns at the tissue-specific sites (e.g., differentially methylated at the particular tissue type relative to other tissue types in the cell-free sample) can be provided as inputs to a ML model that outputs the tissue fraction. The ML model can be trained using training (calibration) samples for which the fractional concentration of the tissue type is known. Such a measurement can be used for various purposes. For example, the fetal DNA fraction in pregnant women can be used for non-invasive prenatal testing (NIPT).

The tissue type can be a clinically-relevant tissue type, e.g., a tissue type that is not normally found in a subject, e.g., fetal tissue, tumor tissue, or transplant tissue. For example, a fraction of liver DNA fragments can be determined for a subject that has undergone a liver transplant. Other transplanted organs can be analyzed in a similar manner.

### A. Fetal Fraction

We further explored whether the nucleosomal patterns could be used to reflect the DNA contribution into plasma from a particular tissue. The pregnancy model was used to address this question. As the placental tissues harbored a great number of unique alleles which were present in placental tissues but absent in background maternal genomes, the placental contribution could be directly deduced using genotype information between the fetal and maternal genomes, providing a gold standard for assessing the nucleosomal pattern-based approach for deducing placental contribution.

We show that a nucleosome signal patterns at tissue-specific CpG sites can be used to accurately determine the tissue fraction of a particular tissue type. This example is performed for fetal DNA to quantitatively measure the fetal DNA fraction, but the analysis is equally applicable to other tissue types.

### 1. Using Nucleosomal Score

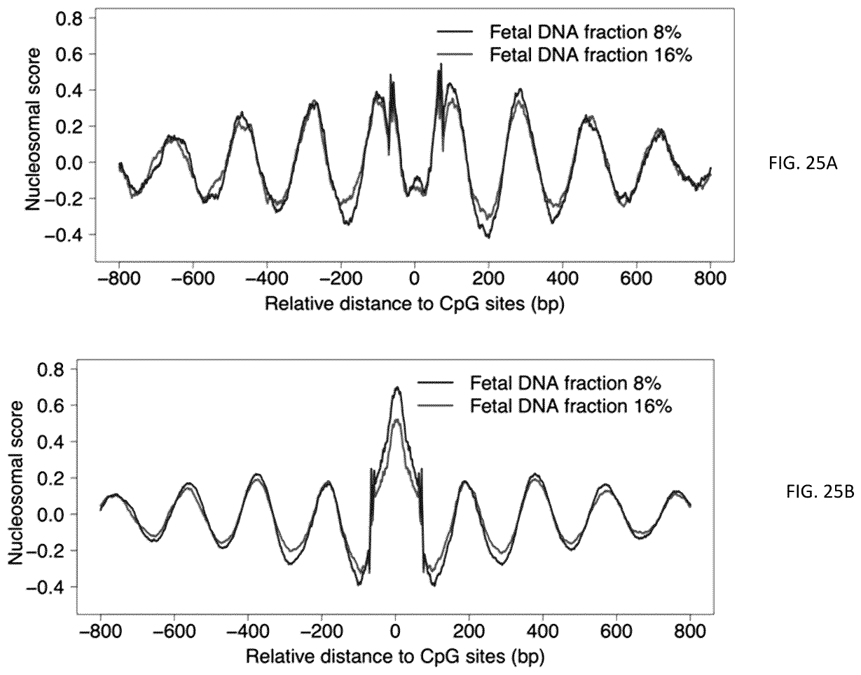

FIGS. 25A and 25B show nucleosomal signal patterns for placenta-specific CpG sites: hypermethylated and hypomethylated. Specifically, FIG. 25A shows nucleosomal score surrounding placenta-specific hypermethylated CpG sites (methylation level<30% in buffy coat and >70% in placenta) using non-bisulfide cfDNA data of pregnant women. But other nucleosome signal patterns can also be used. FIG. 25B shows nucleosomal score surrounding placenta-specific hypomethylated CpG sites (methylation level<30% in placenta and >70% in buffy coat). Similar as the decreased nucleosomal amplitude in cancer patients with higher ctDNA, we observed weakened amplitude in an individual with 16% fetal DNA fraction compared to one with 8%.

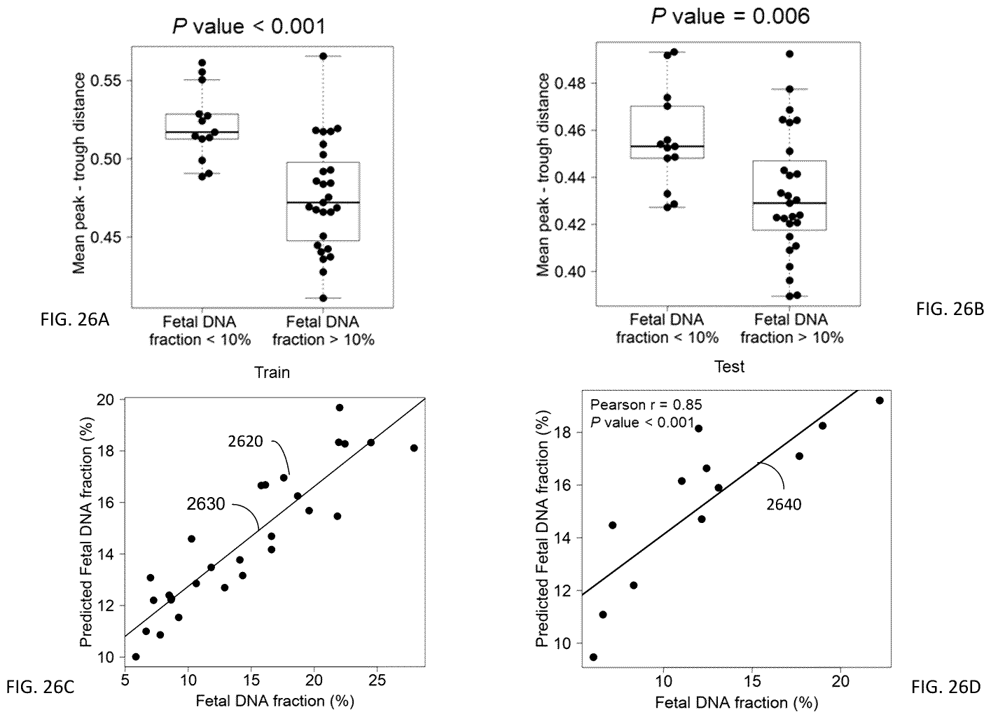

FIG. 26A shows the mean peak-trough distance for hypermethylated sites for different fetal fractions. FIG. 26B shows the mean peak-trough distance for hypomethylated sites for different fetal fractions. As shown, the individuals with higher fetal DNA fraction (>10%) generally showed reduction of nucleosomal amplitude compared to those with lower fraction (<10%) for both hypermethylated (P<0.001, Wilcoxon rank-sum test; FIG. 26A) and hypomethylated (P=0.006, Wilcoxon rank-sum test; FIG. 26B) CpG sites.

Based on a Ridge regression model, we used the nucleosomal scores as independent variables and fetal DNA fraction as dependent variables to construct a linear regression function for fetal DNA fraction prediction.

FIGS. 26C-26D shows fetal DNA fractions predicted by nucleosomal patterns were well correlated with the actual fetal DNA fraction deduced by a single-nucleotide polymorphism (SNP) based approach, with a Pearson's correlation of 0.91 and 0.85 for training and test sets respectively. The results suggested that nucleosomal pattern analysis could be used for deducing the tissue contribution into plasma DNA pool. Other techniques may be used to determine a tissue-specific fraction (e.g., fetal, tumor, or transplant), e.g., using y chromosome for male fetuses or tissue-specific methylation patterns.

The data points 2620 can correspond to calibration data points of calibration samples. The lines 2630 and 2640 correspond to calibration curves (functions) that can be determined from the calibration data points, e.g., by performing a linear regression or non-linear regression. The tissue fraction for a new sample can be determined by comparing the measured nucleosome signal pattern to a reference pattern of any one of the calibration samples. For example, the measured peak-to-trough distance can be compared to a calibration value (peak-to-trough distance for a calibration sample), or the measured peak-to-trough distance can be input to the calibration curve.

2. Using Nucleosomal z-Score

We also show that a nucleosome signal patterns at tissue-specific CpG sites can be used to accurately determine the tissue fraction of a particular tissue type. This example is performed for fetal DNA to quantitatively measure the fetal DNA fraction, but the analysis is equally applicable to other tissue types.

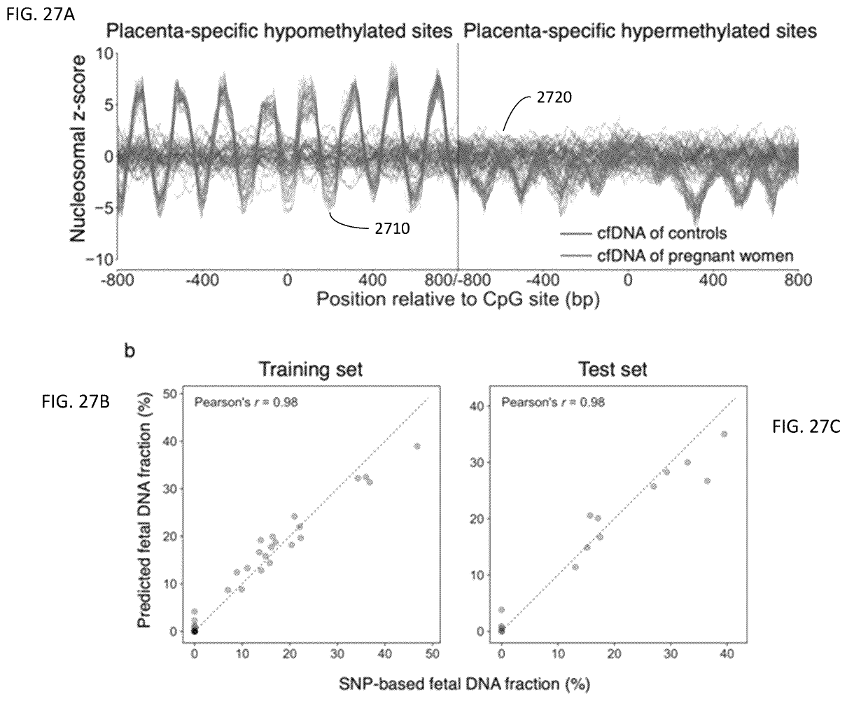

FIG. 27A shows nucleosomal signal patterns for placenta-specific CpG sites: hypomethylated and hypermethylated. Specifically, FIG. 27A shows nucleosomal z-score surrounding placenta-specific CpG sites using cfDNA data of pregnant women 2710 and non-pregnant controls 2720 from Dataset A. But other nucleosome signal patterns can be used.

Based on a Lasso regression model, we used the nucleosomal z-scores as independent variables and fetal DNA fraction as dependent variables to construct a linear regression function for fetal DNA fraction prediction. To determine the nucleosomal z-scores, the mean is of the 19 non-pregnant, healthy controls.

FIGS. 27B-27C show the comparison between actual and predicted fetal DNA fractions in the training and test sets. Actual fractions were deduced from single-nucleotide polymorphism (SNP) analysis. FIG. 27B shows that the fetal DNA fractions predicted by nucleosomal z-scores were well correlated with the actual fetal fraction deduced by a SNP-based approach in a training set comprising 20 pregnant and 14 non-pregnant subjects (Pearson's r: 0.98). FIG. 27C shows that the regression function could be well validated in an independent test set comprising 10 pregnant and 5 non-pregnant subjects, with a Pearson's r of 0.98 between predicted and actual fetal DNA fractions. The results suggest that nucleosomal signal analysis according to the embodiments in this disclosure could be used for noninvasive prenatal testing and deducing the DNA contribution from various tissues.

For the distribution used to determine the z-score, the reference samples might not have DNA fragments from the first tissue type. For example, the reference samples can be from non-pregnant subjects and the first tissue type can be fetal/placental DNA. In other examples, the references samples can have DNA fragments of the first tissue type, but at a normal fractional concentration. For example, the reference samples can have DNA fragments from liver, and the subject being tested can have undergone a liver transplant, where an elevated level of liver DNA fragments is tested.

The regression model that uses the values of a measured nucleosome signal pattern is one example of a machine learning model. Other example models include support vector machine regression, neural networks, or other examples described herein. Such regression models are examples of calibration functions. Thus, the measured nucleosome signal pattern can be input as a feature vector to a machine learning model.

### B. Transplant Fraction

Donor-derived DNA fraction can also be determined, e.g., for liver transplantation recipients or other organ transplant recipients.

We constructed nucleosomal patterns for liver-specific hypermethylated sites (methylation level<30% in buffy coat and >70% in liver tissues) and hypomethylated (methylation level<30% in liver tissues and >70% in buffy coat) CpG sites and examined whether they could be used to estimate donor-derived DNA fraction in liver transplantation recipients. Based on a Ridge regression model, we used the nucleosomal scores as independent variables and donor-derived DNA fraction as dependent variables to construct a linear regression function for donor-derived DNA fraction prediction.

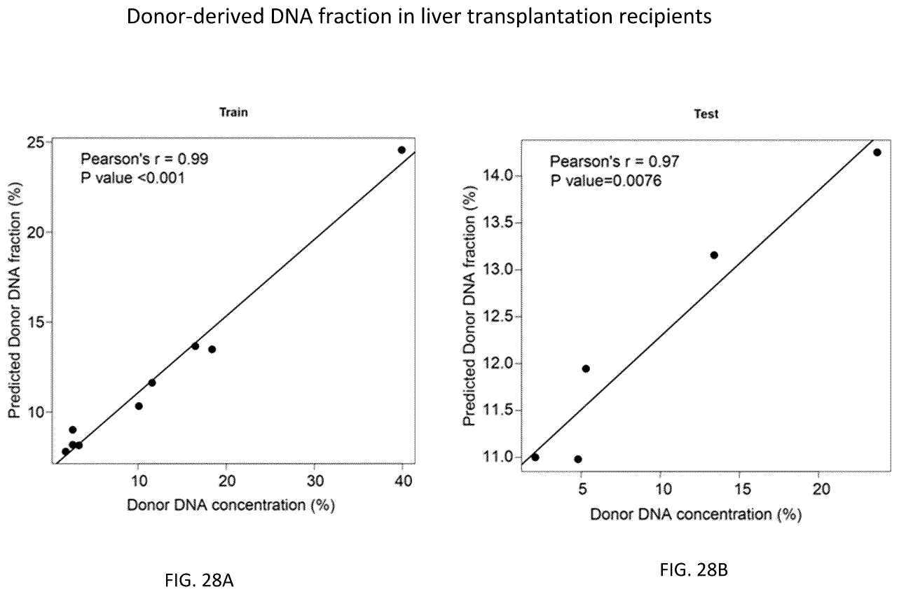

FIGS. 28A-28B shows that donor-derived DNA fractions predicted by nucleosomal patterns were well correlated with the actual donor-derived DNA fraction deduced by single-nucleotide polymorphism analysis, with a Pearson's correlation of 0.99 and 0.97 for training and test sets respectively.

### C. Tumor Fraction

Tumor-derived DNA fraction can also be determined, e.g., for liver cancer such as HCC. DNA fractions from other types of tumors can also be determined, e.g., as other types mentioned herein.

We used a Ridge regression model to measure circulating tumor DNA fraction in plasma samples in Dataset A (Jiang et al., 2020). We used nucleosomal scores to establish a Ridge regression model for predicting tumor DNA fractions in the plasma samples from HCC patients (n=34).

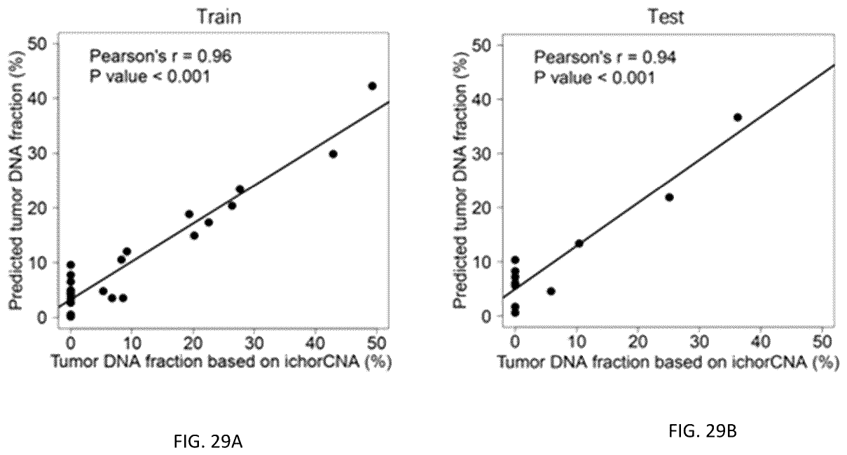

FIGS. 29A-29B shows that tumor-derived DNA fractions predicted by nucleosomal patterns were well correlated with the actual tumor-derived DNA fraction deduced by ichorCNA, with a Pearson's correlation of 0.96 and 0.94 for training and test sets respectively. As shown in FIGS. 29A-29B, tumor DNA fractions predicted by nucleosomal patterns correlated well with those deduced by ichorCNA based on copy number aberration (Adalsteinsson et al., 2017), with a Pearson's correlation of 0.96 and 0.94 for training and test sets, respectively. These results suggested that nucleosomal pattern analysis could be used for deducing the tissue contribution into the plasma DNA pool.

### D. Method for Determining Tissue Fraction

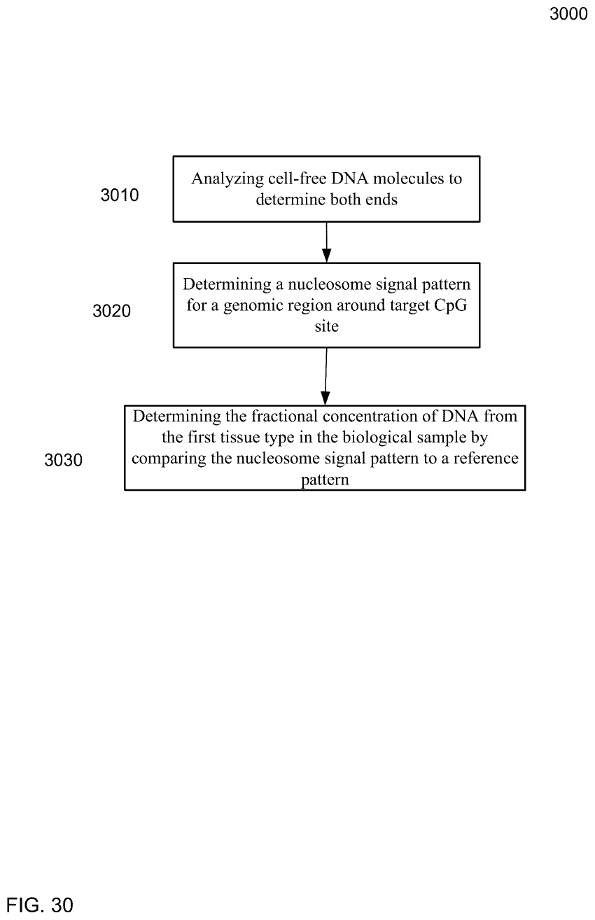

FIG. 30 is a flowchart illustrating a method of determining a fractional concentration of DNA from a first tissue type in a biological sample. Various examples of method 3000 are described above. Method 3000 can be performed partially or entirely using a computer system.

At block 3010, a plurality of cell-free DNA molecules from a biological sample of the subject are analyzed. Block 3010 can be performed in a similar manner as block 610 of method 600 and block 2410 of method 2400.

At block 3020, a nucleosome signal pattern for a genomic region is determined around the target CpG site. Block 3020 can be performed in a similar manner as block 620 of method 600 and block 2420 of method 2400. The target CpG site can be differentially methylated in a target tissue type relative to one or more other tissue types, e.g., as described above. As examples, the biological sample can be plasma or serum, and the one or more other tissue types include blood cells.

In some embodiments, the nucleosome signal pattern can be determined by aggregating over a plurality of genomic regions around a plurality of CpG sites. The plurality of CpG sites can be hypermethylated relative to the one or more other tissue types. The plurality of CpG sites can be hypomethylated relative to the one or more other tissue types. In other examples, both can be used with separate patterns for each.

At block 3030, the fractional concentration of DNA from the first tissue type in the biological sample is determined by comparing the nucleosome signal pattern to a reference pattern. Block 3030 can be performed in a similar manner as block 630 of method 600 and block 2430 of method 2400. The reference pattern can be determined from one or more calibration samples having known fractional concentrations of DNA from the first tissue type.

The reference pattern can be determined from one or more calibration samples having known fractional concentrations of DNA from the first tissue type (also referred to as tissue fraction). The fractional concentration of DNA can have various resolution, e.g., above or below a certain percentage or within a range. Such range can have various resolutions, such as 5%, 10%, 15%, 20%, 25%, and 30% resolution or less. As an example, a 5% resolution can correspond to 35%-40%.

The tissue fraction for a new sample can be determined by comparing to a reference pattern of any one of the calibration samples or as an input to the calibration curve (e.g., using a peak to trough distance). When the one or more calibration samples are a plurality of calibration samples, the comparing of the nucleosome signal pattern to the reference pattern can include inputting a peak-to-trough distance of the nucleosome signal pattern into a calibration function. The calibration function can be determined by measuring the fractional concentrations of DNA from the first tissue type for a plurality of calibration samples and measuring peak-to-trough distances of the plurality of calibration samples, thereby determining calibration data points comprising the fractional concentrations and the peak-to-trough distances. The calibration function can then be fit to the calibration data points, e.g., using regression.

In other examples, determining the fractional concentration of DNA from the first tissue type can include using each of the nucleosome signals, e.g., via a comparison to a respective signal of a reference pattern, as may occur when a machine learning model is used. Such nucleosome signal values can be used in a feature vector for a model. Thus, comparing each of the signal values to the reference pattern can be performed by inputting the nucleosome signal pattern as part of a feature vector into a machine learning model, e.g., a regression model or other type described herein. Accordingly, determining the fractional concentration of DNA from the first tissue type can include inputting the nucleosome signal pattern into a machine learning model that was trained using reference patterns of a plurality of calibration samples.

The subject and first tissue type can have various forms. For example, the subject can be pregnant with a fetus, and the first tissue can be fetal tissue. As another example, the first tissue type is from a particular organ.

As a further step, a level of cancer can be determined in the first tissue type of the subject based on the fractional concentration.

### V. Treatments

### A. Further Screening Modalities

Based on any classification, e.g., regarding a pathology or fractional concentration of clinically-relevant DNA, the subject can be referred for additional screening modalities, e.g. using chest X-ray, ultrasound, computed tomography, magnetic resonance imaging, or positron emission tomography. Such screening may be performed for cancer.

### B. Treatment Selection

Embodiments of the present disclosure can accurately predict disease relapse, thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contribute to cancer growth and survival. For example, erdafitinib is a drug given orally that is approved to treat people with locally advanced or metastatic urothelial carcinoma with FGFR3 or FGFR2 genetic mutations that have continued to grow or spread cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### VI. Example Systems

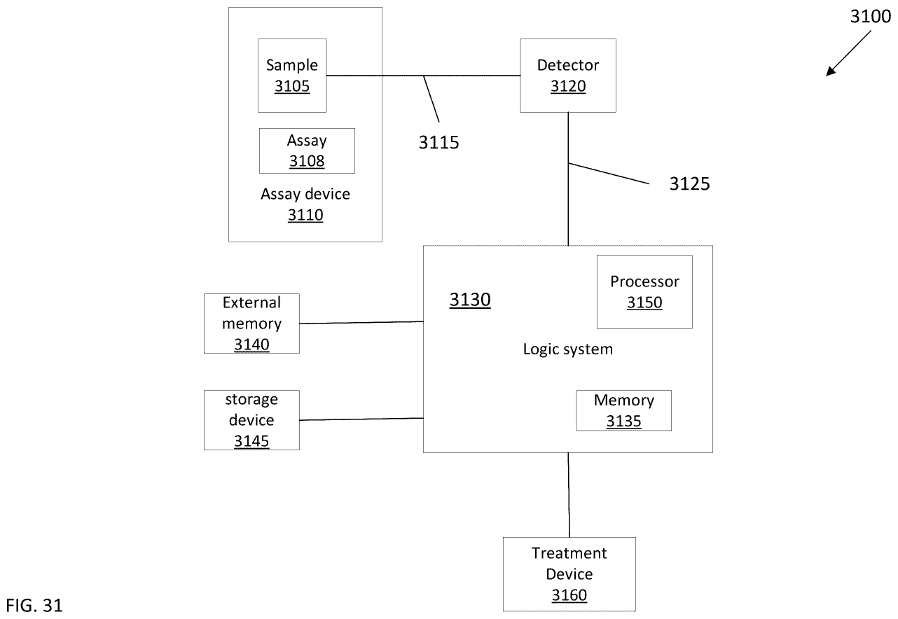

FIG. 31 illustrates a measurement system 3100 according to an embodiment of the present disclosure. The system as shown includes a sample 3105, such as cell-free nucleic acid molecules (e.g., DNA and/or RNA) within an assay device 3110, where an assay 3108 can be performed on sample 3105. For example, sample 3105 can be contacted with reagents of assay 3108 to provide a signal of a physical characteristic 3115 (e.g., sequence information of a cell-free nucleic acid molecule). An example of an assay device can be a flow cell that includes probes and/or primers of an assay or a tube through which a droplet moves (with the droplet including the assay). Physical characteristic 3115 (e.g., a fluorescence intensity, a voltage, or a current), from the sample is detected by detector 3120. Detector 3120 can take a measurement at intervals (e.g., periodic intervals) to obtain data points that make up a data signal. In one embodiment, an analog-to-digital converter converts an analog signal from the detector into digital form at a plurality of times.

Assay device 3110 and detector 3120 can form an assay system, e.g., a sequencing system that performs sequencing according to embodiments described herein. A data signal 3125 is sent from detector 3120 to logic system 3130. As an example, data signal 3125 can be used to determine sequences and/or locations in a reference genome of nucleic acid molecules (e.g., DNA and/or RNA). Data signal 3125 can include various measurements made at a same time, e.g., different colors of fluorescent dyes or different electrical signals for different molecule of sample 3105, and thus data signal 3125 can correspond to multiple signals. Data signal 3125 may be stored in a local memory 3135, an external memory 3140, or a storage device 3145. The assay system can be comprised of multiple assay devices and detectors.

As an example usable in any of the embodiments described above, sample procession and sequencing of cell-free DNA (e.g. plasma DNA) can be performed as follows. DNA was extracted from 1.6 mL of plasma from each sample using the QIAamp Circulating Nucleic Acid Kit (QIAGEN) according to the manufacturer's protocol. Indexed plasma DNA libraries were constructed using TruSeq Nano DNA Library Prep kits (Illumina) and xGen UDI-UMI adaptors (Integrated DNA Technologies) according to the manufacturer's instructions. Multiplexed DNA libraries were sequenced using a pair-end mode (75 bp×2) on a NovaSeq 6000 platform (Illumina). Sequence read alignment was performed using the Short Oligonucleotide Alignment Program 2 (SOPAP2) (21), and paired-end reads were filtered to remove misaligned and duplicated reads. Genomic DNA was extracted from 200-400 μL of maternal buffy coat samples using the QIAamp DNA Blood Mini Kit (QIAGEN) according to the manufacturer's protocol. Maternal genotype information was obtained from microarray analysis using Infinium Omni2.5 BeadChip (Illumina). Fetal DNA fraction was determined using FetalQuantSD (22).

Logic system 3130 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 3130 and the other components may be part of a stand-alone or network connected computer system, or they may be directly attached to or incorporated in a device (e.g., a sequencing device) that includes detector 3120 and/or assay device 3110. Logic system 3130 may also include software that executes in a processor 3150. Logic system 3130 may include a computer readable medium storing instructions for controlling measurement system 3100 to perform any of the methods described herein. For example, logic system 3130 can provide commands to a system that includes assay device 3110 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 3100 may also include a treatment device 3160, which can provide a treatment to the subject. Treatment device 3160 can determine a treatment and/or be used to perform a treatment. Examples of such treatment can include surgery, radiation therapy, chemotherapy, immunotherapy, targeted therapy, hormone therapy, and stem cell transplant. Logic system 3130 may be connected to treatment device 3160, e.g., to provide results of a method described herein. The treatment device may receive inputs from other devices, such as an imaging device and user inputs (e.g., to control the treatment, such as controls over a robotic system).

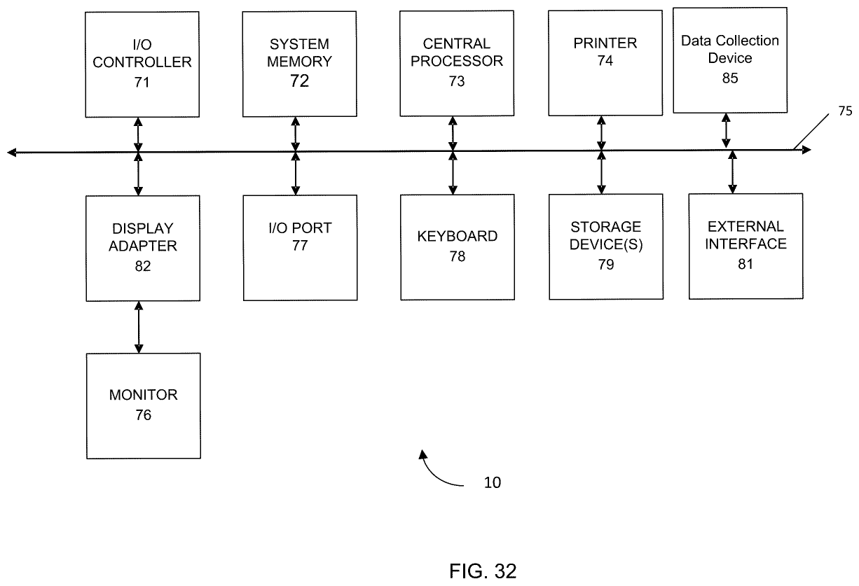

Any of the computer systems mentioned herein may utilize any suitable number of subsystems. Examples of such subsystems are shown in FIG. 32 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 32 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

What is claimed is:

1. A method of analyzing a biological sample to determine a level of a cancer in the biological sample of a subject, the biological sample including cell-free DNA, the method comprising:
analyzing a plurality of cell-free DNA molecules from the biological sample of the subject, wherein analyzing each of the plurality of cell-free DNA molecules includes determining two genomic positions in a reference genome corresponding to both ends of the cell-free DNA molecule;
determining a nucleosome signal pattern for a genomic region around a target CpG site by:
for each genomic position within the genomic region:
determining a first amount of the plurality of cell-free DNA molecules that span a window around the genomic position, the window being two bp or greater in length;
determining a second amount of the plurality of cell-free DNA molecules that end within the window around the genomic position; and
determining a per-position nucleosome signal using the first amount and the second amount, wherein the target CpG site is differentially methylated in a target tissue type relative to one or more other tissue types; and
determining a classification of the level of the cancer for the subject based on a comparison of the nucleosome signal pattern to a reference pattern, wherein the reference pattern is determined from one or more training samples having a known level of the cancer, and wherein the level of the cancer is determined for the target tissue type.

2. The method of claim 1, wherein for at least one of the one or more training samples, the known level of the cancer is that the subject does not have the cancer.

3. The method of claim 1, wherein the determining the classification of the level of the cancer for the subject includes inputting the nucleosome signal pattern into a machine learning model that was trained using the reference pattern of the one or more training samples.

4. The method of claim 1, wherein the subject has a viral infection, and wherein the classification of the level of the cancer is that cancer does not exist but that the subject is at a higher risk for cancer than other subjects having the viral infection.

5. The method of claim 1, wherein the classification of the level of the cancer is a likelihood of the subject getting cancer in future.

6. The method of claim 5, further comprising:
comparing the likelihood to a threshold; and
performing monitoring of the subject based on the likelihood exceeds the threshold.

7. The method of claim 6, wherein the subject is monitored by performing screening at a higher rate when the likelihood exceeds the threshold than is performed when the likelihood is less than the threshold.

8. The method of claim 1, wherein the target tissue type is of an organ.

9. The method of claim 1, wherein the comparison of the nucleosome signal pattern to the reference pattern uses a peak-to-trough distance.

10. The method of claim 1, wherein the biological sample is plasma or serum, and wherein the one or more other tissue types include blood cells.

11. The method of claim 1, wherein the nucleosome signal pattern is determined by aggregating over a plurality of genomic regions around a plurality of CpG sites.

12. The method of claim 11, wherein the plurality of CpG sites are hypermethylated relative to the one or more other tissue types.

13. The method of claim 11, wherein the plurality of CpG sites are hypomethylated relative to the one or more other tissue types.

14. The method of claim 1, wherein the genomic region is at least 140 bp in length.

15. The method of claim 1, wherein the per-position nucleosome signal incudes a difference or a ratio of the first amount and the second amount.

16. The method of claim 1, wherein the per-position nucleosome signal is normalized using nucleosome signals from other genomic regions.

17. The method of claim 16, wherein the normalization uses a mean value or a median value of per-position nucleosome signals derived from the other genomic regions.

18. The method of claim 16, wherein the other genomic regions are adjacent to the genomic region.

19. The method of claim 1, wherein the nucleosome signal pattern is normalized using a region-level statistical value determined from one or more regions, resulting in the per-position nucleosome signal being a nucleosome score.

20. The method of claim 19, wherein the one or more regions include the genomic region or one or more other regions.

21. The method of claim 20, wherein the one or more regions include the one or more other regions that are adjacent to the genomic region.

22. The method of claim 19, wherein the region-level statistical value is a mean value or a median value.

23. The method of claim 1, wherein the per-position nucleosome signal is normalized using a background signal that is dependent on a distance of the genomic position to the target CpG site.

24. The method of claim 23, wherein the background signal is determined from one or more other genomic regions, each centered around a CpG site.

25. The method of claim 24, wherein the one or more regions are selected randomly.

26. The method of claim 1, wherein the per-position nucleosome signal is normalized using a distribution of reference nucleosome signals determined from one or more regions of one or more references samples.

27. The method of claim 26, wherein normalizing using the distribution includes:
for each position, determining an aggregate statistical value and a dispersion value of the distribution of reference nucleosome signals; and
subtracting the aggregate statistical value from each per-position nucleosome signal and dividing by the dispersion value.

28. The method of claim 27, wherein the distribution is the normal distribution, wherein the aggregate statistical value is a mean of the reference nucleosome signals of the position, and wherein the dispersion value is a standard deviation.

29. The method of claim 1, wherein the window is at least 5 bp in length.

30. The method of claim 29, wherein the window is at least 50 bp in length.

31. The method of claim 30, wherein the window is at least 100 bp in length.

32. The method of claim 1, wherein analyzing the plurality of cell-free DNA molecules includes sequencing the plurality of cell-free DNA molecules to obtain sequence reads.

33. The method of claim 32, wherein determining the two genomic positions in the reference genome corresponding to both ends of the cell-free DNA molecule comprises aligning the sequence reads to the reference genome.

34. A method for measuring methylation of a target CpG site in a genome of a subject using cell-free DNA molecules, the method comprising:
analyzing a plurality of cell-free DNA molecules from a biological sample of the subject, wherein analyzing each of the plurality of cell-free DNA molecules includes determining two genomic positions in a reference genome corresponding to both ends of the cell-free DNA molecule;
determining a nucleosome signal pattern for a genomic region around the target CpG site by:
for each genomic position within the genomic region:
determining a first amount of the plurality of cell-free DNA molecules that span a window around the genomic position, the window being two bp or greater in length;
determining a second amount of the plurality of cell-free DNA molecules that end within the window around the genomic position; and
determining a per-position nucleosome signal using the first amount and the second amount, wherein the genomic region is at least 140 bp in length; and
determining a methylation level of the target CpG site in the genome of the subject based on a comparison of the nucleosome signal pattern to a reference pattern, wherein the reference pattern is determined from one or more training samples having a known methylation level.

35. The method of claim 34, wherein the methylation level is that the target CpG site is hypermethylated or hypomethylated.

36. The method of claim 34, the methylation is a range for a methylation density at the target CpG site.

37. The method of claim 34, wherein the biological sample is plasma or serum, wherein the target CpG site is known to be differentially methylated between a first tissue type and blood cells, and wherein the methylation level is determined for the target CpG site in the first tissue type.

38. The method of claim 37, wherein the first tissue type is cancer tissue.

39. The method of claim 37, wherein the first tissue type is fetal tissue or of a particular organ.

40. The method of claim 37, wherein the target CpG site is known to be differentially methylated between the first tissue type and the blood cells by having a difference in methylation of at least 30%.

41. The method of claim 34, wherein the target CpG site is in a center of the genomic region.

42. A method for measuring a fractional concentration of DNA from a first tissue type in a biological sample of a subject, the biological sample comprising cell-free DNA, the method comprising:
analyzing a plurality of cell-free DNA molecules from the biological sample of the subject, wherein analyzing each of the plurality of cell-free DNA molecules includes determining two genomic positions in a reference genome corresponding to both ends of the cell-free DNA molecule;
determining a nucleosome signal pattern for a genomic region around a target CpG site by:
for each genomic position within the genomic region:
determining a first amount of the plurality of cell-free DNA molecules that span a window around the genomic position, the window being two bp or greater in length;
determining a second amount of the plurality of cell-free DNA molecules that end within the window around the genomic position; and
determining a per-position nucleosome signal using the first amount and the second amount, wherein the target CpG site is differentially methylated in the first tissue type relative to one or more other tissue types in the biological sample; and
determining the fractional concentration of DNA from the first tissue type in the biological sample by comparing the nucleosome signal pattern to a reference pattern, wherein the reference pattern is determined from one or more calibration samples having known fractional concentrations of DNA from the first tissue type.

43. The method of claim 42, wherein subject is pregnant with a fetus, and wherein the first tissue type is fetal tissue.

44. The method of claim 42, wherein the first tissue type is from a particular organ.

45. The method of claim 42, wherein the target CpG site is differentially methylated in the first tissue type relative to one or more other tissue types in the biological sample by having a difference in methylation level of at least 30%.

46. The method of claim 42, further comprising:
determining a level of cancer in the first tissue type of the subject based on the fractional concentration of DNA from the first tissue type in the biological sample.

47. The method of claim 42, wherein the one or more calibration samples are a plurality of calibration samples, wherein determining the fractional concentration of DNA from the first tissue type includes inputting the nucleosome signal pattern into a machine learning model that was trained using reference pattern of the plurality of calibration samples.

48. The method of claim 42, wherein the one or more calibration samples are a plurality of calibration samples, and wherein comparing the nucleosome signal pattern to the reference pattern includes inputting a peak-to-trough distance of the nucleosome signal pattern into a calibration function.

49. The method of claim 48, wherein the calibration function is determined by:
measuring fractional concentrations of DNA from the first tissue type for the plurality of calibration samples;
measuring peak-to-trough distances of the plurality of calibration samples, thereby determining calibration data points comprising the fractional concentrations and the peak-to-trough distances; and
fitting the calibration function to the calibration data points.
